(19) United States
(12) Patent Application Publication
(10) Pub. No.: US 2005/0288218 A1
(43) Pub. Date: Dec. 29, 2005

(54) Methods for treating and preventing sepsis using modified C1 inhibitor or fragments thereof

(75) Inventors: Alvin E. Davis, Boston, MA (US); Dongxu Liu, Brookline, MA (US)

Correspondence Address: LAHIVE & COCKFIELD, LLP., 28 STATE STREET, BOSTON, MA, 02109, US

(73) Assignee: CBR Institute for Biomedical Research, Boston, MA (US)

(21) Appl. No.: 11/090,915

(22) Filed: Mar. 24, 2005

Related U.S. Application Data
(63) Continuation of application No. PCT/US03/30630, filed on Sep. 25, 2003.
(60) Provisional application No. 60/413,341, filed on Sep. 25, 2002.

(51) Int. Cl. A61K 38/17

(52) U.S. Cl. 514/8

(57) ABSTRACT

The present invention is based, at least in part, on the discovery that the amino terminal domain and the N-linked carbohydrate contained in the amino terminal of C1INH are required for binding of C1INH to LPS. C1INH has the ability to block the binding of LPS to cells, e.g., macrophages. One aspect of the invention provides a method for treating or preventing sepsis in a subject comprising administering to the subject an effective amount of a composition comprising a modified C1INH polypeptide, thereby treating or preventing sepsis in a subject. In another aspect, the invention provides a method for treating or preventing LPS-mediated inflammation in a subject comprising administering to the subject an effective amount of a composition comprising a modified C1INH polypeptide, thereby treating or preventing LPS-mediated inflammation in a subject. In yet another aspect, the invention provides a method for suppressing the release of LPS-induced TNF-α in a subject comprising administering to the subject an effective amount of a composition comprising a modified C1INH polypeptide, thereby suppressing the release of LPS-induced TNF-α in a subject.

### RELATED APPLICATIONS

This application is a continuation of PCT/US2003/030630, entitled “Methods for Treating and Preventing Sepsis Using Modified C1 Inhibitor or Fragments Thereof,” filed on Sep. 25, 2003, which claims the benefit of U.S. Provisional Patent Application No. 60/413,341, entitled “Methods for Treating and Preventing Sepsis Using Modified C1 Inhibitor or Fragments Thereof,” filed on Sep. 25, 2002. The entire contents of these applications are hereby incorporated herein by reference.

### GOVERNMENT RIGHTS

This invention was made at least in part with government support under grant nos. HD22082 and HD33727, awarded by the National Institutes of Health. The government has certain rights in this invention.

### BACKGROUND OF THE INVENTION

Bacterial sepsis and related septic shock are frequently lethal conditions caused by infections which can result from certain types of surgery, abdominal trauma and immune suppression related to cancer, transplantation therapy, or other disease states. It is estimated that over 700,000 patients become susceptible to septic shock-causing bacterial infections each year in the United States alone. Of these, 160,000 actually develop septic shock, resulting in 50,000 deaths annually.

Gram negative bacterial infections comprise the most serious infectious disease problem seen in modern hospitals. Two decades ago, most sepsis contracted in hospitals was attributable to more acute gram positive bacterial pathogens such as Staphylococcus and Streptococcus. By contrast, the recent incidence of infection due to gram negative bacteria, such as, for example, Escherichia coli and Pseudomonas aeruginosa, has increased.

Gram negative bacteria now account for some 200,000 cases of hospital-acquired infections yearly in the United States, with an overall mortality rate in the range of 20% to 80%. The majority of these hospital-acquired infections are due to such gram negative bacilli as E. coli (most common pathogen isolated from patients with gram negative sepsis), followed in frequency by Klebsiella pneumoniae and P. aeruginosa.

Gram negative sepsis is a disease syndrome resulting from the systemic invasion of gram negative rods and subsequent endotoxemia caused by release of endotoxin, the lipopolysaccharide (LPS) moiety of the organisms' cell walls, into the circulation. The severity of the disease ranges from a transient, self-limiting episode of bacteremia to a fulminant, life-threatening illness often complicated by organ failure and shock. The disease is often the result of invasion from a localized infection site, or may result from trauma, wounds, ulcerations or gastrointestinal obstructions. The symptoms of gram negative sepsis include fever, chills, pulmonary failure and septic shock. Septic shock is characterized by hypotension and organ dysfunction (Parillo, J. E. (1993) The New England Journal of Medicine 328(2):1471).

Gram negative infections are particularly common among patients receiving anti-cancer chemotherapy and immunosuppressive treatment. Infections in such immuno-compromised hosts characteristically exhibit resistance to many antibiotics, or develop resistance over the long course of the infection, making conventional treatment difficult. The ever increasing use of cytotoxic and immunosuppressive therapy and the natural selection for drug resistant bacteria by the extensive use of antibiotics have contributed to gram negative bacteria evolving into pathogens of major clinical significance. C1-esterase inhibitor (also known as C1-INH or C1INH) is a protease inhibitor present in the plasma. The C1INH from human plasma consists of a single glycosylated polypeptide chain, with a molecular weight of about 105 000 daltons. C1INH is the only inhibitor of the classical complement pathway proteases, C1r and C1s (Sim, et al. (1979) FEBS Lett 97:111), and is the major inhibitor of factor XII (FXIIa), factor XI (factor XIa), and prekallikrein of the contact system (de Agostini, et al. (1984) J. Clin Investigation 93:1542; Schapira, et al. (1982) J. Clin Invest 69:462). As a major effector of inflammation, the complement system has been implicated in both the pathogenesis of and protection from lipopolysaccharide (LPS)-induced shock (Caliezi, et al. (2001) Pharmacol. Rev 52:91). Similarly, the contact system also appears to play a role in the mediation of symptoms in septic shock (Colman, et al. (1999) Thromb Haemost 82:1568).

LPS is composed of two chemically dissimilar structural regions: the hydrophilic repeating polysaccharide of the core and O-antigen structures and a hydrophobic domain known as lipid A (LPA) (Ulevitch, R. J. (1995) Annu Rev Immunol. 13:437-457). LPA is the toxic principle of Gram-negative bacterial LPS and has full endotoxin activity (Grabarek, J., G. et al. (1990) J Biol Chem. 265:8117-8121; Takada, H., and S. Kotani (1989) CRC Crit Rev Microbiol. 16:477-423). Virtually all LPS-induced biological responses are LPA dependent (Rietschel, E. T. et al. (1994) FASEB J 8:217-225). Therefore, recognition of LPA by cells must be the initial step in LPS-induced cellular responses. The general chemical structure of LPA from diverse gram-negative bacteria is highly conserved (Ulevitch, R. J. (1995) Annu Rev Immunol. 13:437-457). LPA has the biological function to induce nuclear factor-KB activation in monocytes (Mansell, A. et al. (2001) FEBS Lett. 508:313-317) and the production of proinflammatory cytokines such as tumor necrosis factor-a (TNF-α) and interleukin-1 from macrophages (Astiz, M. E., (1995) Crit Care Med. 23:9-17, Henrieson, B. E. et al. (1993) Infect Immun. 61:2325-2333).

The LPS-binding protein (LBP), an acute phase reactant present in blood interacts with and transfers endotoxin LPS to CD14. CD14, the primary receptor for LPS, exists in a soluble form in blood and as a GPI-linked molecule on the surface of mononuclear phagocytes (Fenton, M. J. & Golenbock, D. T. J Leukocyte Biol (1998) 64:25-32; Poltorak, A. et al. (1998) Science 282:2085-2088; Schumann, R. R. & Latz, E. (2000) Chem Immunol 74:42-60; and Kitchens, R. L. (2000) Chem Immunol 74). LPS-induced mononuclear phagocytes release a variety of potent inflammatory mediators including TNF-α (Nagaoka, I. et al. (2001) J Immunol 167:3329-3338), leading to sepsis and septic shock. Although C1INH is an acute phase protein, its antigenic levels tend to be normal in patients with fatal septic shock, while levels of proteolytically inactivated C1INH are increased, which suggests an increased turnover and a relative secondary deficiency of biologically active C1INH during sepsis (Nuijens, J. H. et al. (1989) J Clin Invest 84:443-450). The biological effects, if any, of inactivated C1INH remain unknown. Beneficial effects of C1INH have been observed in several animal models of sepsis (Caliezi, C. et al. (2001) Pharmacol. Rev. 52:91-112; Triantaphyllopoulos, D. C. & Cho, M. S. (1986) Thrombosis & Haemostasis 55:293; Guerrero, R. et al. (1993) J Clin Invest 91:2754-2760; Scherer, R. U., et al. (1996) Seminars in Thrombosis & Hemostasis 22:357-366; Fischer, M. B. et al. (1997) J Immunol 159:976-982; and Jansen, P. M. et al. (1998) J Immunol 160:475-484). However, the precise mechanism(s) of C1INH protection in endotoxemia remains ill-defined.

Based on the life threatening nature of sepsis and the lack of effective treatment or prevention, it would be beneficial to identify prophylactic and therapeutic compositions and methods useful for the treatment and prevention of sepsis.

### SUMMARY OF THE INVENTION

The present invention is based, at least in part, on the discovery of a novel anti-inflammatory function of C1INH that is unrelated to its previously identified protease inhibitory activity. In one embodiment, the present invention is based on the discovery that modified C1INH polypeptides specifically, e.g., directly, bind to LPS. In one embodiment, the modified C1INH polypeptide is an amino terminal fragment of a C1INH polypeptide. In another embodiment, the amino terminal fragment is glycosylated, e.g., contains at least one N-linked carbohydrate. In another embodiment, the modified C1INH polypeptide has reduced protease inhibition activity. The binding of LPS to macrophages induces the release of a variety of potent inflammatory mediators including TNF-α, thus leading to sepsis and inflammation in a subject. Accordingly, a modified C1INH polypeptide, e.g., a modified C1INH polypeptide having reduced protease inhibition activity, binding to and inhibiting LPS, thereby preventing or and treating sepsis and inflammation in a subject.

Furthermore, the invention is based, at least in part, on the discovery that N-deglycosylation significantly reduces C1INH-mediated protection of mice from lethal LPS-induced shock. In addition, N-deglycosylated C1INH does not bind to LPS, does not inhibit the binding of LPS to RAW 264.7 cells or to human blood cells, and does not prevent the activation of these cells by LPS. Furthermore, it has been found that C1INH binding to LPS is mediated via the hydrophobic domain of LPS known as lipid A (LPA) and that this binding also is reduced by removal of N-linked carbohydrate from C1INH. Accordingly, the invention is based, at least in part, on the discovery that N-linked glycosylation of C1INH is required for the interaction of C1INH with endotoxin.

Accordingly, one aspect of the invention provides a method for treating or preventing sepsis in a subject comprising administering to the subject an effective amount of a composition comprising a modified C1INH polypeptide, e.g., a modified polypeptide which contains intact N-linked carbohydrate, e.g., N-linked carbohydrate linked to the amino terminal domain of C1INH, or a fragment thereof which is capable of binding LPS, thereby treating or preventing sepsis in a subject.

In another aspect, the invention provides a method for treating or preventing LPS-mediated inflammation in a subject comprising administering to the subject an effective amount of a composition comprising a modified C1INH polypeptide, thereby treating or preventing LPS-mediated inflammation in a subject.

In yet another aspect, the invention provides a method for suppressing the release of LPS-induced TNF-α in a subject comprising administering to the subject an effective amount of a composition comprising a modified C1INH polypeptide, thereby suppressing the release of LPS-induced TNF-α in a subject.

In still another aspect, the invention provides a method of inhibiting the binding of LPS to a cell, e.g., a macrophage, in a subject comprising administering to the subject an effective amount of a composition comprising a modified C1INH polypeptide, thereby inhibiting the binding of LPS to a cell, e.g., a macrophage, in a subject.

In one embodiment, the subject is a mammal, e.g., a human. In another embodiment, the composition comprises a pharmaceutically acceptable carrier.

In one embodiment, the modified C1INH polypeptide specifically binds LPS. In another embodiment, the modified C1INH polypeptide lacks an intact serpin reactive loop. In yet another embodiment, the modified C1INH polypeptide comprises at least one mucin-like domain. In a further embodiment, the modified C1INH polypeptide comprises at least one tetrapeptide sequence comprising an amino acid sequence Glx-Pro-Thr-Thr. In another embodiment, the modified C1INH polypeptide comprises less than seven tetrapeptide sequence. In yet another embodiment, the modified C1INH polypeptide comprises seven tetrapeptide sequences.

In a further embodiment, the modified C1INH polypeptide lacks substantially lacks protease inhibition activity. In another embodiment, the modified C1INH polypeptide comprises the amino terminal domain of C1INH, e.g., amino acids 23-119 of C1INH, or an active fragment thereof In another embodiment, the modified polypeptide contains intact N-linked carbohydrate, e.g., N-linked carbohydrate linked to amino acids 23-119 of C1INH , or an active fragment thereof. In still another embodiment, the amino terminal domain comprises intact N-linked carbohydrate at, e.g., Asn residues 25, 69, and/or 81 of the C1INH protein sequence (SEQ ID NO:2). In yet another embodiment, the modified C1INH polypeptide specifically binds LPS but does not substantially inhibit activation of the complement system. In still another embodiment, the modified C1INH polypeptide specifically binds LPS but does not substantially inhibit activation of the contact system.

In another aspect, the invention provides a method for modulating the binding of LPS to a macrophage comprising contacting LPS with an agent which specifically binds LPS but does not substantially inhibit activation of the complement system, thereby modulating the binding of LPS to a macrophage. In one embodiment, the agent comprises a modified C1INH polypeptide. In another embodiment, the modified C1INH polypeptide lacks an intact serpin reactive loop of C1INH. In still another embodiment, the modified C1INH polypeptide lacks substantially lacks protease inhibition activity. In yet another embodiment, the agent comprises the amino terminal domain of C1INH, e.g., amino acids 23-119 of C1INH, or an active fragment thereof. In another embodiment, the modified polypeptide contains intact N-linked carbohydrate, e.g., linked to amino acids 23-119 of C1INH, or an active fragment thereof. In still another embodiment, the amino terminal domain comprises intact N-linked carbohydrate at, e.g., Asn residues 25, 69, and/or 81 of the C1INH protein sequence (SEQ ID NO:2). In another embodiment, the agent is a small molecule.

In yet another aspect, the invention provides a method for treating or preventing sepsis in a subject comprising administering to said subject a composition comprising an agent which specifically binds LPS but does not substantially inhibit activation of the complement system, thereby modulating the binding of LPS to a macrophage. In one embodiment, the composition further comprises a pharmaceutically acceptable carrier.

In still another aspect, the invention provides a composition comprising an agent, e.g., a small molecule which specifically binds LPS and does not inhibit activation of the complement system or the contact system. In one embodiment, the composition further comprises a pharmaceutically acceptable carrier.

Other features and advantages of the invention will be apparent from the following detailed description and claims.

### BRIEF DESCRIPTION OF THE DRAWINGS

FIG. 1 is a graph depicting the effect of C1INH and cleaved C1INH on survival of mice in Gram-negative endotoxin LPS-induced lethal endotoxemia. C57BL/6J mice were injected with either LPS (20 mg/kg)(i.p.) following treatment with C1INH (200 μg/per mouse) i.p. (n=20) (filled squares) or i.v. (n=20)(filled circles), with cleaved C1INH (200 μg/per mouse) i.v. (n=10)(open circles), or with a mixture of LPS (20 mg/kg) and C1INH (200 μg/per mouse) i.p.(n=20)(filled triangle) or cleaved C1INH i.p. (n=10)(open triangle). As the controls, mice were injected with LPS (20 mg/kg) i.p. (n=16) alone (filled diamond) or with C1INH (200 μg/per mouse) i.v. (n=4) alone (open diamond).

FIGS. 2A-F depict the effect of C1INH on the binding of FITC-conjugated LPS to RAW 264.7 macrophages. A. The binding of LPS to macrophages in the presence of C1INH (10 to 300 μg/ml). B. The binding of LPS to macrophages preincubated with C1INH. C. Anti-C1INH antibody reverses the inhibitory effect of C1INH on LPS binding to macrophages. BSA did not interfere with C1INH-mediated inhibition of LPS binding to macrophages. D. Effect of C1INH on the binding of LPS to macrophages analyzed by fluorescence microscopy. E and F. C1lNH-mediated inhibition of LPS-induced TNF-α mRNA production in macrophages.

FIGS. 3A-B depict the effect of C1INH-C1s complexes and of cleaved C1INH on the binding of LPS to RAW 264.7 macrophages. A. C1INH (150 μg/ml) was incubated with C1s (150 and 300 μg/ml) to form C1H-C1s complexes. Macrophages were incubated with LPS (175 ng/ml) in the presence of C1INH-C1s complexes. B. C1INH (150 to 600 μg/ml) was incubated with trypsin. Macrophages were incubated with LPS (175 ng/ml) and the cleaved C1INH.

FIGS. 4A-E depict the interaction of C1INH with LPS. A: Binding of C1INH to immobilized LPS either alone (filled diamond), or in the presence of added BSA (filled squares) or IgG (filled triangles). Controls consisted of BSA (open squares), IgG (open triangles) or PBS (open diamonds) added to LPS coated plates in the absence of C1INH. B. FBS competed with binding of C1INH to LPS. C. Human LBP peptide (5-40 ng/ml) competed with binding of C1INH to LPS. D. LPS alters the electrophoretic mobility of C1INH. E. LPS has no effect on C1INH-C1s complex formation as assessed by SDS-PAGE.

FIG. 5A-B depicts the effect of truncated C1INH on LPS binding. A. LPS binding to RAW 264.7 macrophages is not inhibited by treatment with truncated C1INH. B. Truncated C1INH did not bind to LPS.

FIG. 6 depicts the nucleotide and amino acid sequence of C1INH (SEQ ID NOs:1 and 2, respectively). The C1INH protein contains a signal peptide of 22 residues in the amino-terminal end which is cleaved resulting in a 478 amino acid mature protein. The amino terminal domain of C1INH contains 7 repeats of the tetrapeptide sequence Glx-Pro-Thr-Thr, or variants thereof, which are indicated by boxes.

FIG. 7 depicts the results of SDS-PAGE analysis of deglycosylated C1INH. C1INH (10 μg) was incubated with N-glycosidase F, O-glycosidase, and neuraminidase as described in Example 2. The various forms of C1INH were analyzed by SDS-PAGE and stained with Coomassie brilliant blue.

FIG. 8 is a graph depicting the effect of deglycosylated C1INH on survival of mice in Gram-negative endotoxin LPS-induced lethal endotoxemia. C57BL/6J mice were injected i.p. with a mixture of LPS (20 mg/kg) with native, plasma-derived C1INH (open triangles, n=20), with N-deglycosylated C1INH (filled diamonds, n=10), with 0-deglycosylated C1INH (filled triangles, n=10), or with N— and O-deglycosylated C1INH (filled squares, n=10) (200 μg/per mouse). Control mice were injected (i.p.) with LPS (20 mg/kg) alone (open circles, n=16) or a mixture of LPS (20 mg/kg) with the glycosidase buffer (open squares, n=5). The indicated P values are for each group in comparison with the group treated with native, plasma derived C1INH.

FIG. 9A-C depicts the effect of deglycosylated C1INH on the binding of FITC-conjugated LPS to the murine macrophage cell line, RAW 264.7. LPS binding, thick line; control, shaded field. (A), RAW 264.7 macrophages were incubated with FITC-LPS (175 ng/ml) in the absence or presence of C1INH, 7V-deglycosylated C1INH, O-deglycosylated C1INH, or N— and O-deglycosylated C1INH (each at 150 μg/ml) in DMEM containing 10% FBS for 15 minutes at 37° C. (B), RAW 264.7 macrophages were incubated with FITC-LPS (175 ng/ml) in the presence of different doses of 0-deglycosylated C1INH (150, 75, 37.5, 10, and 5 μg/ml) in DMEM containing 10% FBS for 15 minutes at 37° C. (C), RAW 264.7 macrophages were incubated with FITC-LPS (175 ng/ml) in the presence of iCIINH (150 μg/ml) lacking N—, O—, or N— and O-linked glycosylation. Cells were fixed with FACS solution after washing with PBS three times and were analyzed by FACS.

FIG. 10 depicts the effect of deglycosylated C1INH on the binding of FITC-conjugated LPS to human blood cells. LPS binding, thick line; control, shaded field. The human blood cells were incubated for 15 minutes at 37° C. with FITC-LPS (175 ng/ml) in the presence of C1INH (150 (μg/ml) lacking N—, O—, or N— and O-linked glycosylation. Cells were fixed with FACS solution after washing with PBS three times and were analyzed by FACS.

FIG. 11A-B depicts the effect of deglycosylated C1INH on LPS-induced TNF-α mRNA expression in the murine macrophage cell line, RAW 264.7 and human blood cells. (A), total RNA from RAW 264.7 macrophages was isolated after treatment with LPS (175 ng/ml) in the presence of C1INH or with N—, O—, or N— and O-deglycosylated C1INH (all at 150 μg/ml) for 30 minutes at 37° C. RT-PCR was performed using mouse TNF-α cDNA and β-actin cDNA primers. (B), total RNA from human blood cells was isolated after treatment of whole human blood with LPS (175 ng/ml) in the presence of C1INH (150 μg/ml) or with N—, O—, or N— and O-deglycosylated C1INH for 30 minutes at 37° C. RT-PCR was performed using human TNF-α cDNA and β-actin cDNA primers.

FIG. 12A-C are graphs depicting the results of an analysis of the binding of deglycosylated C1INH to LPS and LPA. The interaction of C1INH or deglycosylated C1INH with immobilized LPS, mLPA, dLPA, and dLPS was analyzed by ELISA. (A), binding of deglycosylated C1INH (150 μg/ml) to LPS (50, 100, and 150 ng/ml). (B), binding of C1INH (150 μg/ml) to mLPA (0.5, 1.0, and 5.0 μg/ml), dLPA (0.5, 1.0, and 5.0 μg/ml), and dLPS (0.5, 1.0, and 5.0 Hg/ml). (C), binding of deglycosylated C1INH (150 μg/ml) to mLPA (1.0 μg/ml), dLPA (1.0 μg/ml), and dLPS (1.0 μg/ml).

FIG. 13A-B depicts the effects of mLPA, dLPA, and dLPS on the formation of C1INH-C1s complexes and C1INH cleavage by trypsin. (A), mLPA (0.1, 0.2, 0.5, and 1.0 μg), dLPA (0.1, 0.2, 0.5, and 1.0 g), and dLPS (0.1, 0.2, 0.5, and 1.0 ng) have no effect on the rate or extent of C1INH-C1s (10 μg: 10 μg) complex formation, as assessed by SDS-PAGE stained with Coomassie brilliant blue. (B), mLPA (0.5, 25, and 50 ng), dLPA (0.1, 0.5, 25, and 50 ng), and dLPS (0.1, 0.5, 25, and 50 ng) have no effect on the formation of cleaved C1INH (10 μg) by trypsin, as assessed by SDS-PAGE stained with Coomassie brilliant blue.

### DETAILED DESCRIPTION OF THE INVENTION

The present invention is based, at least in part, on the discovery of a novel anti-inflammatory function of C1INH that is unrelated to its previously identified protease inhibitory activity, e.g., the inhibition of the activation of the complement system through inhibition of C1, C1r, or C1s, or the inhibition of the activation of the contact system through inhibition of kallikrein, factor XIa, or factor XIIa. It has been found that modified C1INH directly interacts with, e.g., specifically binds, endotoxin LPS. The binding of LPS to cells, e.g., macrophages, induces the release of a variety of potent inflammatory mediators including TNF-α, thus leading to sepsis in a subject. C1INH, e.g., modified C1INH, specifically binds to LPS and modulates, e.g., inhibits, LPS binding to cells, e.g., macrophages, therefore suppressing the release of LPS induced inflammatory mediators and treating and/or preventing sepsis.

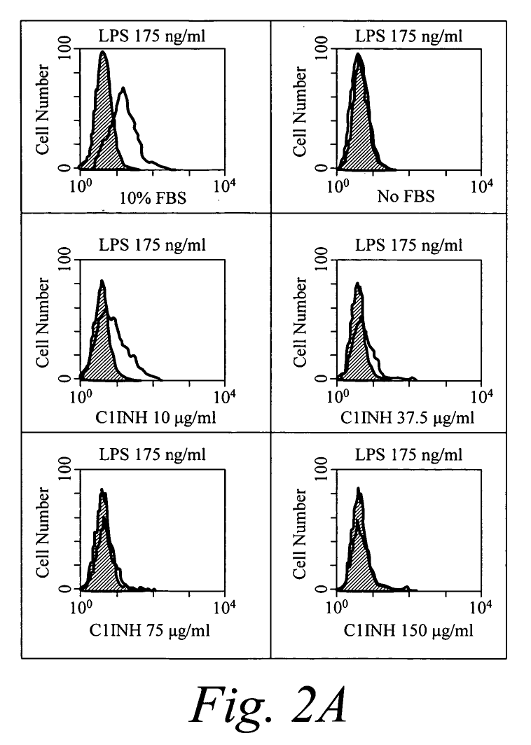

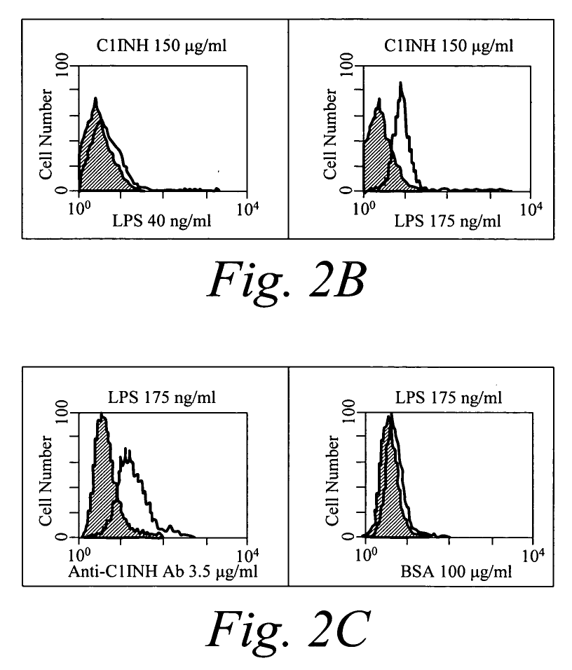

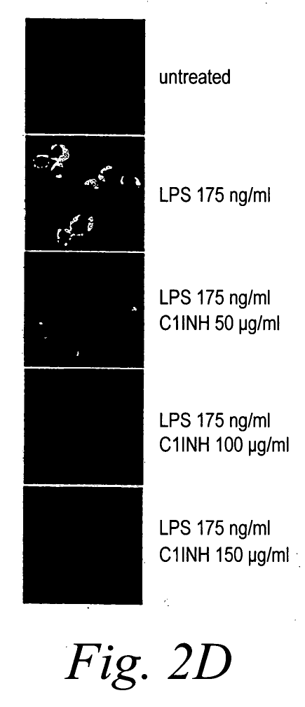

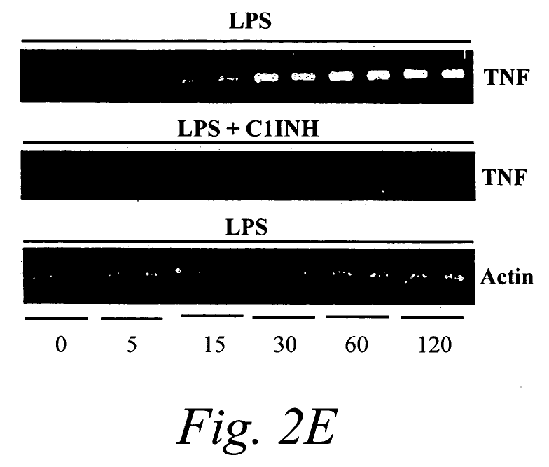

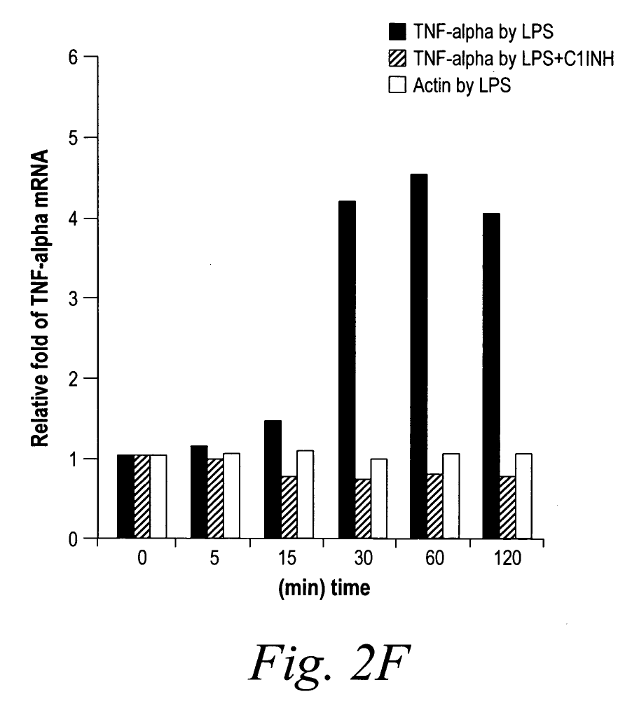

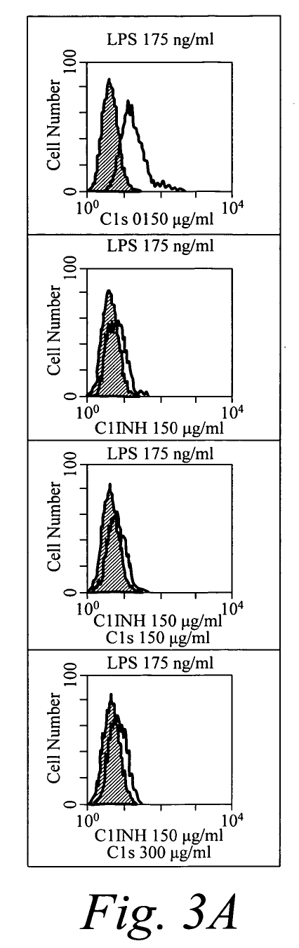

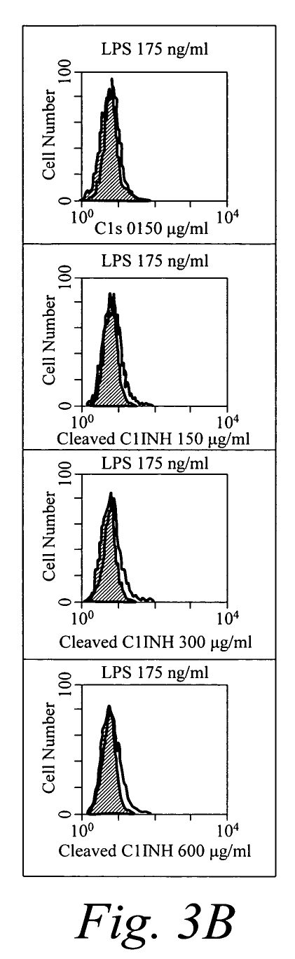

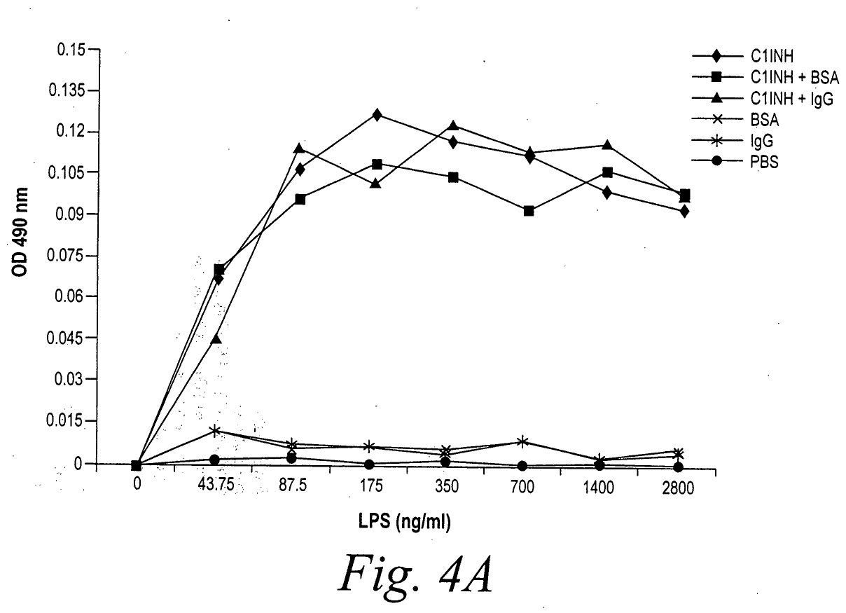

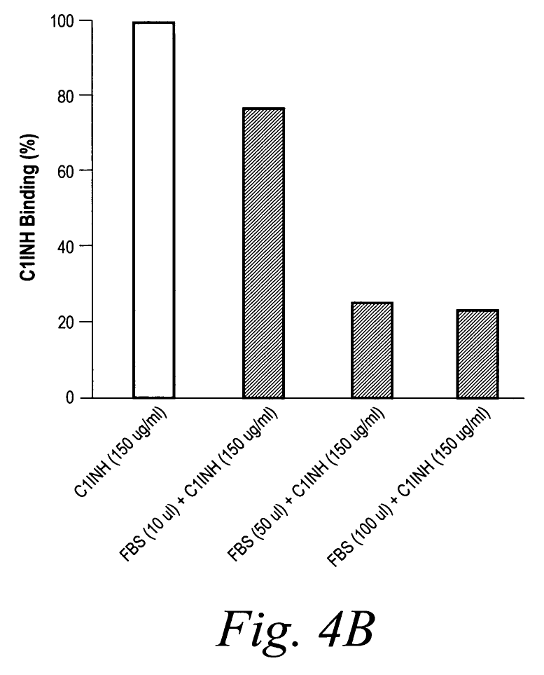

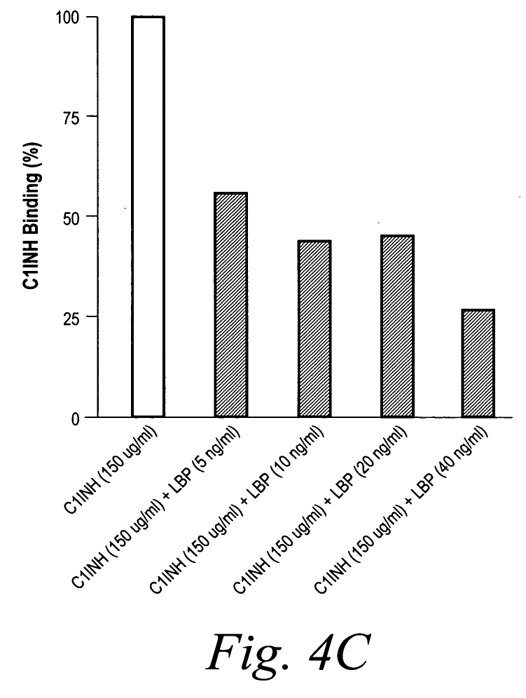

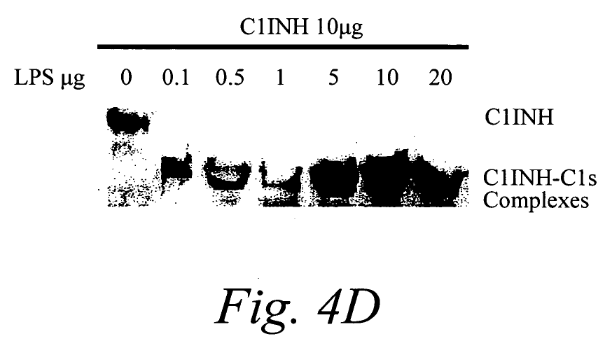

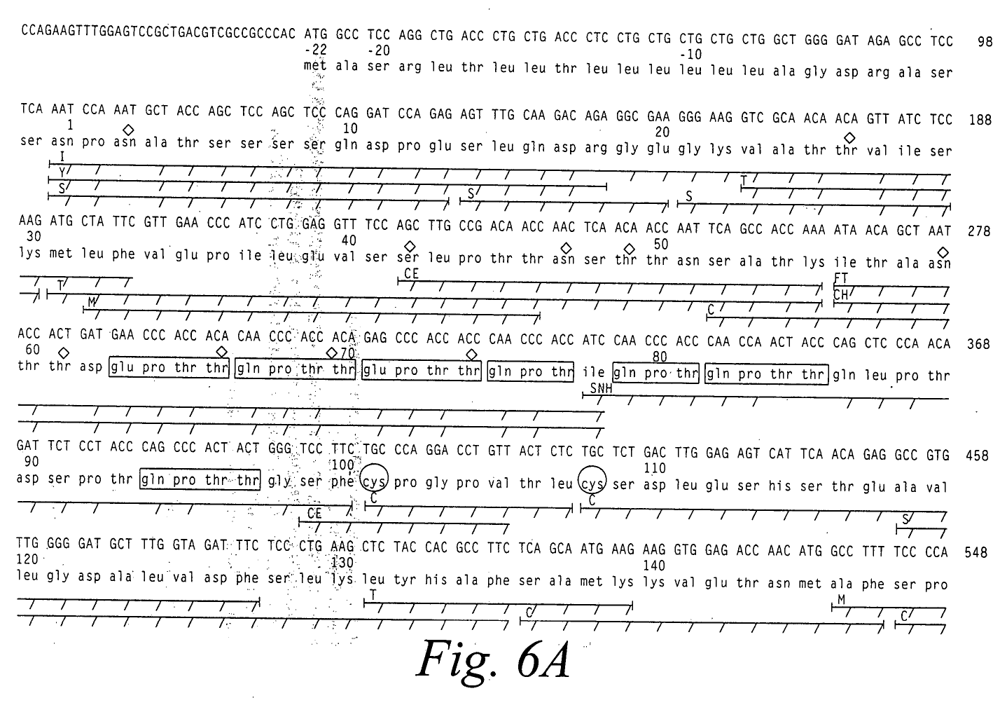

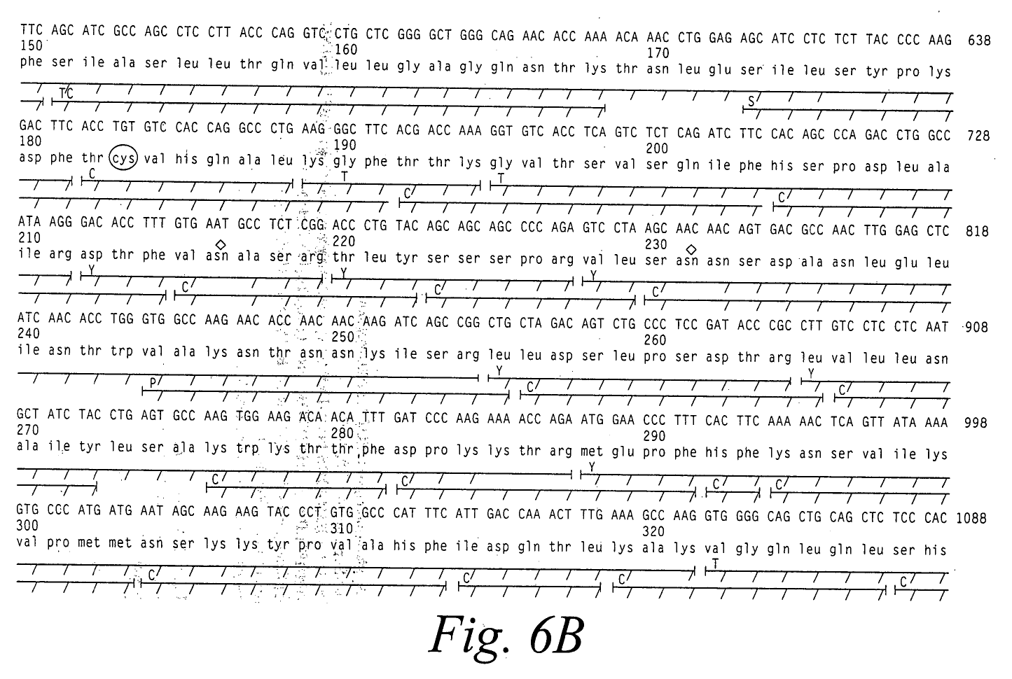

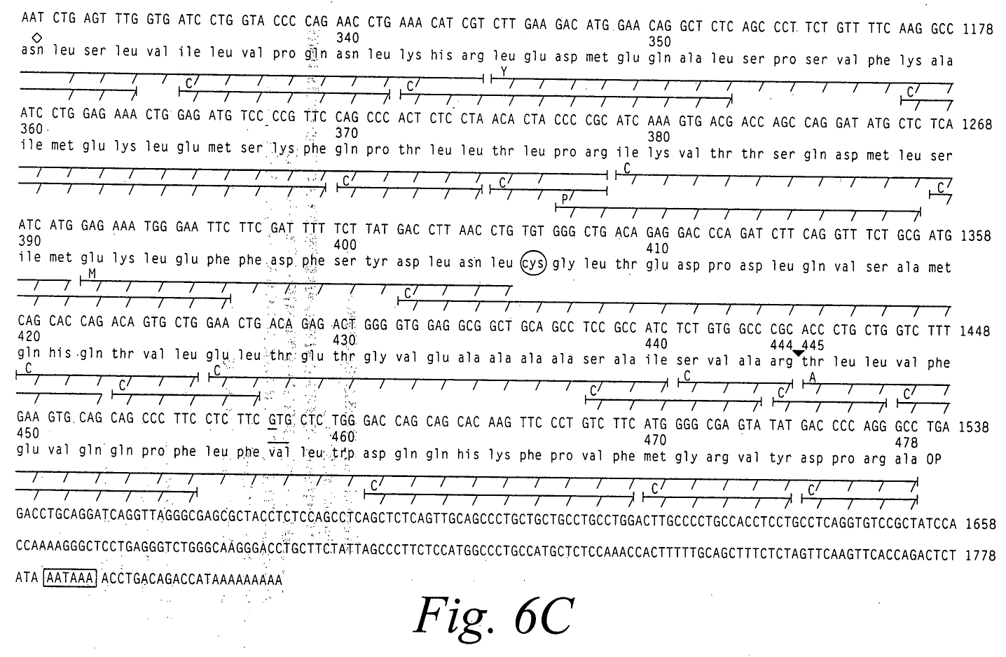

Furthermore, it has been found that N-deglycosylation significantly reduces C1INH-mediated protection of mice from lethal LPS-induced shock (FIG. 2). In addition, N-deglycosylated C1INH does not bind to LPS, does not inhibit the binding of LPS to RAW 264.7 cells (FIG. 3) or to human blood cells (FIG. 4), and does not prevent the activation of these cells by LPS. In addition, CIINH binding to LPS is mediated via LPA, and this binding also is reduced by removal of N-linked carbohydrate from CIINH. Lastly, it has been discovered that the direct binding of C1INH to LPS is dependent on N-glycosylation (FIG. 6A, Table 1). Accordingly, the invention is based, at least in part, on the discovery that N-linked glycosylation of C1INH is required for the interaction of C1INH with endotoxin LPS.

Reactive center cleaved, inactive CIINH (referred to herein as iCHNH), which has no protease inhibitory activity, retains the ability to bind to LPS, but that binding requires the presence of the amino terminal non-serpin domain (Table 1). A C1INH polypeptide with deletion of the amino terminal 119 amino acids retains the ability to inactivate proteases but does not bind to LPS (Table 1) (Liu, et al. (2003) J. of Immunology 171:2594-2601, incorporated herein by reference). Deglycosylation of C1INH with N-glycanase, O-glycanase, or both, has no significant effect on protease inhibitory function of C1INH (Reboul, A. et al. (1987) Biochem J. 24:117-121, incorporated herein by reference). Therefore, the three N-linked carbohydrates within the amino terminal domain of C1INH at Asn residues 25, 69 and 81, and not those within the serpin domain, are required for reactivity with endotoxin. However, the binding site on the C1INH amino terminal domain does not reside on the N-linked carbohydrate itself, but on the peptide backbone. Therefore, the C1INH amino terminal domain and intact N-linked carbohydrate are required for C1INH binding to endotoxin.

TABLE 1
The binding of CIINH to LPS^{a}

| | LPS | dLPA | mLPA | dLPS |
|---|---|---|---|---|
| CIINH | +^{b} | + | + | − |
| iCHNH | + | ND | ND | ND |
| Truncated C1INH^{C} | − | ND | ND | ND |
| N-deglycosylated CIINH | − | ± | − | − |
| O-deglycosylated CIINH | + | + | + | − |
| N- + O-deglycosylated CIINH | − | ± | − | − |

^{a}The table summarizes data from FIG. 6.
^{b}(+), strong binding; (±), intermediate binding; (−), weak or no binding; ND, not determined.
^{c}Truncated CIINH, recombinant CIINH with deletion of the amino-terminal 119 amino acid residues.

The active component of endotoxin, LPA, is a phosphoglycolipid having an acylated and phosphorylated dihexosamine headgroup. The polysaccharide component contains antigenic determinants but does not contribute to endotoxin activity (Ribi, E. 1986. Structure-function relationship of bacterial adjuvants, p. 35. In R. M. Nervig, P. M. Gough, M. L. Kaeverle, and C. A. Whetstone (ed.), Advances in Carries and Adjuvants for Veterinary Biologicals. Iowa State University Press, Ames; Rietschel, E. T., T. (1994) FASEB J. 8:217-225). The removal of an acid labile phosphate group and normal fatty acid groups from dLPA diminishes endotoxic activity. Although mLPA lacks many of the endotoxic properties of LPS (Astiz, M. E., R. C. Rackow, J. G. Still, S. T. Howell, A. Cato, K. B. Von Eschen, J. T. Ulrich, J. A. Rudbach, G. McMahon, R. Vargas, and W. Stern (1995) Crit Care Med. 23:9-17), in vitro it induces the production of proinflammatory cytokines from macrophages (Henrieson, B. E., et al. (1993) Infect Immun. 61:2325-2333) and interferon-γ and IL-2 from lymphocytes (Carozzi, S., (1989) J Clin Microbiol. 27:1748-1753). However, mLPA is markedly less active than is LPS in the induction of these cytokines. In addition, mLPA and LPS may differentially regulate the production of some cytokines. For example, mLPA induced IL-10 to a greater extent than did LPS (Salkowski, C. A., et al. 1997 Infect Immun. 65:3239-3247). In addition, mLPA activates human dendritic cells and T cells and enhances the generation of both Th1- and Th2-specific immune responses in mice (De Becker, G., V (2000) Int Immunol. 12:807-815; Ismaili, J., (2002) J Immunol. 168:926-932).

C1INH interacts with the LPA moiety within LPS to prevent its interaction with LBP and, ultimately, with cells, e.g., macrophages. It was found that C1INH at a fixed concentration (150 μg/ml) bound to immobilized DLPA, but did not bind as efficiently to mLPA, and bound extremely poorly to dLPS (FIG. 6B). As with binding to LPS, the binding of C1INH to LPA also was reduced by N-deglycosylation, but not by removal of O-linked carbohydrate (FIG. 6C). However, the reduction in binding of N-deglycosylated versus native C1INH to dLPA (FIG. 6C) was not as striking as the reduction in binding of N-deglycosylated C1INH to LPS (FIG. 6A). Without intending to be bound by theory, these findings indicate that the binding site on the C1INH amino terminal domain does not reside on the N-linked carbohydrate itself, but is on the peptide backbone. Removal of carbohydrate may result in alteration of the conformation of this domain and partially mask the binding site. The smaller dLPA molecule may be able to more readily gain access to the binding site while the larger LPS molecule would be unable to interact with the site. Binding of LPS (or dLPA) to many LPS-binding proteins is via interaction of the phosphate groups on the LPA with specific positively charged residues within the LPS-binding protein. C1INH has one Arg (at position 40) and 3 Lys residues (at positions 44, 52 and 77) within the amino terminal domain.

Therefore, the binding of C1INH to LPS prevents the interaction of LPS with LBP which in turn prevents the delivery of LPS to cells that express the LPS receptor complex. The characteristics of this binding are that it is mediated by the LPA moiety of LPS, it does not require an intact C1INH reactive center loop (and therefore is not dependent on protease inhibitory activity), but does require the C1INH amino terminal non-serpin domain and intact N-linked carbohydrate.

As used herein, the term “intact N-linked carbohydrate” or “N-linked glycosylation” refers to one or more N-linked carbohydrates which are capable of mediating an interaction between C1INH, e.g., modified C1INH, and LPS. For example, N-linked carbohydrates include the three N-linked carbohydrates within the amino terminal domain of C1INH at Asn residues 25, 69 and 81 of the C1INH protein sequence (SEQ ID NO:2; GenBank Accession No. P05155) which are required for binding of C1INH to LPS. In one embodiment, all three N-linked carbohydrates are present within the amino terminal domain of C1INH at Asn residues 25, 69 and 81 of the C1INH protein sequence (SEQ ID NO:2).

Accordingly, in one aspect, the present invention is directed to the use of modified C1INH polypeptides which specifically bind LPS for the treatment and prevention of sepsis. In one embodiment, a modified C1INH polypeptide comprises the amino terminal domain of C1INH. In one embodiment, the amino terminal domain comprises intact N-linked carbohydrate, e.g., at Asn residues 25, 69, and/or 81 of the C1INH protein sequence (SEQ ID NO:2). In another embodiment, a modified C1INH polypeptide does not have an intact serpin reactive center loop. In another embodiment, deletion of the amino terminal 119 amino acid residues abrogates the ability of C1INH to interact with LPS. Moreover, in another embodiment, cleaved, inactive C1INH, e.g., C1INH which is unable to act as a protease inhibitor because it lacks an intact center reactive loop, referred to herein as iC1INH, acts to prevent or treat sepsis in a subject. Thus, modified C1INH polypeptides which contain the amino terminal domain, e.g., amino acids 23-119 of C1INH, which, e.g., comprises intact N-linked carbohydrate, can be used to treat or prevent sepsis in a subject. In another embodiment, fragments of C1INH which contain the amino terminal domain, e.g., amino acids 23-119 of C1INH, which, e.g., comprise intact N-linked carbohydrate, can also be used to modulate LPS-mediated inflammation. In a further embodiment, fragments of C1INH which contain the amino terminal domain, e.g., amino acids 23-119 of C1INH, which, e.g., comprise intact N-linked carbohydrate can also be used to suppress the release of LPS-induced TNF-α or other inflammatory mediators from cells, e.g., macrophages.

In another aspect, the invention provides a method for modulating the binding of LPS to a cell, e.g., a macrophage, comprising contacting LPS with a composition comprising an agent which specifically binds to LPS but does not substantially inhibit the complement system, e.g., by inhibition of C1, C1r, or C1s, thereby modulating the binding of LPS to cell, e.g., a macrophage. In one embodiment, the agent does not substantially inhibit the complement system.

In still another aspect, the invention provides a method for modulating the binding of LPS to a cell, e.g., a macrophage, comprising contacting LPS with a composition comprising an agent which specifically binds LPS but does not substantially inhibit the contact system, e.g., by inhibition of kallikrein, factor XIa, or factor XIIa, thereby modulating the binding of LPS to a cell, e.g., a macrophage. In one embodiment, the agent does not substantially inhibit the contact system.

As used herein, the phrase “reduced or substantially eliminated protease inhibitory activity” means that the protease inhibitory activity of a protease inhibitor, e.g., C1INH, or a modified C1INH, is reduced. That is, while there may be some protease inhibitor activity, inhibition of proteases, e.g., C1, C1r, or C1s or kallikrein, factor XIa, or factor XIIa, is not carried out to the fullest extent.

As used herein, the phrase “does not substantially inhibit activation of the complement system or contact system” means that inhibition of activation of the complement or contact system is inhibited to some extent but may not be completely inhibited. Inhibition of the activation of the complement system or the contact system can be assayed for by identifying the presence of SDS-stable enzyme-inhibitor complexes and proteolytically cleaved C1INH (see, e.g., Schapira et al. (1988) Methods Enzymol 163:179-185, incorporated herein by reference).

Administration of C1INH, e.g., modified C1INH, or a fragment thereof, e.g., a fragment containing intact N-linked carbohydrate, to a subject for the treatment or prevention of sepsis may be alone or in combination with other agents known to aid in the treatment or prevention of sepsis, e.g., antibiotics, anti-TNF-α antibodies, and/or anti-LPS antibodies, which may be produced as described in U.S. Pat. No. 6,315,999, the contents of which are incorporated herein by reference. In another embodiment, when therapeutically beneficial, C1INH, or a fragment thereof, may be administered in combination with any agent which acts as a protease inhibitor to inhibit the complement system, e.g., through inhibition of C1, C1s, or C1r, and/or any agent which inhibits contact system activation, e.g., through inhibition of plasma kallikrein, factor XIa, or factor XIIa, for example. Administration of C1INH and another agent may be serially or as a mixture.

“Sepsis” or “endotoxemia,” used interchangeably herein, is defined as any disease or disorder, including inflammation or inflammatory disease or disorder resulting from gram-positive or gram-negative bacterial infection, the latter primarily due to the bacterial endotoxin liposaccaride (LPS). Sepsis can be induced by at least the six major gram-negative bacilli which are: Pseudomonas aeriginosa, Escherichia coli, Proteus, Klebsiella, Enterobacter and Serratia. Sepsis also includes septic shock, and any disease or disorder related to resulting from septic shock, including, for example, hypotension, oliguria, tachycardia, tachypnea, and fever.

“C1INH” is defined herein as a plasma glycoprotein with a molecular weight of about 105 000 that belongs to the superfamily of serine protease inhibitors. It is known to inhibit activated components of the classical pathway of complement, C1, C1r, and C1s, and the intrinsic contact system, factor XIa, factor XIIa, and kallikrein.

C1INH contains an amino terminal domain which is a mucin-like domain and is heavily glycosylated with three N-linked and at least seven O-linked carbohydrate groups (Bock et al. (1986) Biochemistry 25:4292-4301, the contents of which are incorporated by reference). The amino terminal domain comprises amino acids 23-142 of C1INH. In one embodiment, the N-terminal domain of C1INH comprises amino acids 23-119 of C1INH. The domain contains up to 7 repeats of the tetrapeptide sequence Glx-Pro-Thr-Thr, or variants thereof (see FIG. 6) (see Bock et al. (1986) Biochemistry 25:4292-4301 and Coutino, et al. (1994) J. Immunol 153:3648-3654, the contents of which are incorporated herein by reference).

C1INH also contains a “serpin domain” (also referred to herein as a “serpin reactive center loop,” or a “reactive loop”), comprising amino acid residues 142 through the C-terminus of C1INH (see Bock et al. (1986), supra). The serpin domain is glycosylated with three N-linked groups. An intact, e.g., functional, unmodified serpin reactive domain is essential for protease inhibitory activity of C1INH.

A “modified C1INH polypeptide,” or “modified C1INH protein,” as used interchangeably herein, includes any C1INH polypeptide or protease inhibitor which substantially lacks the protease inhibitory activity of naturally occurring C1INH or has reduced or substantially eliminated protease inhibitory activity as compared to naturally occurring C1INH. In one embodiment, a modified C1INH polypeptide is a C1INH polypeptide, or active fragment thereof, which specifically binds to or inhibits LPS but does not substantially inhibit activation of the complement pathway, e.g., through inhibition of C1, C1r, or C1s, for example. In another embodiment, a modified C1INH inhibitor is a C1INH polypeptide which specifically binds to or inhibits LPS but does not substantially inhibit activation of the contact pathway, e.g., through inhibition of plasma kallikrein, factor XIa, or factor XIIa, for example. In one embodiment, a modified C1INH polypeptide lacks an intact serpin reactive center loop domain. In another embodiment, a modified C1INH polypeptide has a modified or cleaved serpin reactive center loop domain that has reduced or substantially eliminated protease inhibitory activity, e.g., a fragment which does not comprise an intact serpin domain or comprises a serpin domain that has been mutated or cleaved, e.g. resulting in inactive C1INH (iCHNH). In another embodiment, a modified C1INH polypeptide comprises at least one mucin-like domain which, e.g., comprises at least one intact N-linked carbohydrate e.g., at Asn residues 25, 69, and/or 81 of the C1INH protein sequence (SEQ ID NO:2). In yet another embodiment, a modified C1INH polypeptide comprises one or more, preferably 2, 3, 4, 5, 6, or up to 7 tetrapeptide sequences. In another embodiment, a modified C1INH polypeptide comprises an amino terminal domain of C1INH which is a heavily glycosylated mucin-like domain comprising amino acids 23-142 of C1INH. In yet another embodiment, a modified C1INH polypeptide comprises the amino terminal domain of C1INH, e.g., amino acids 23-119. In one embodiment, the amino terminal domain comprises intact N-linked carbohydrate, e.g., at Asn residues 25, 69, and/or 81 of the C1INH protein sequence (SEQ ID NO:2). In yet another embodiment, a modified C1INH polypeptide comprises a fragment of the C1INH polypeptide.

A “fragment of C1INH” as used herein, includes a polypeptide which comprises less than the full-length polypeptide and which retains to ability to specifically bind to or inhibit LPS. In one embodiment, the fragment comprises intact N-linked carbohydrate e.g., at Asn residues 25, 69, and/or 81 of the C1INH protein sequence (SEQ ID NO:2). In one embodiment, the fragment lacks an intact serpin reactive center loop. In another embodiment, the fragment comprises at least one mucin-like domain. In yet another embodiment, the fragment comprises one or more, preferably 2, 3, 4, 5, 6, or up to 7 tetrapeptide sequences. In still another embodiment, the fragment comprises amino acids 23-119 of C1INH which, e.g., comprises intact N-linked carbohydrate, e.g., at Asn residues 25, 69, and/or 81 of the C1INH protein sequence (SEQ ID NO:2), or an active fragment thereof. In another embodiment, a fragment of C1INH comprises a portion of the C1INH polypeptide which specifically binds to or inhibits endotoxin LPS, but does not function as a protease inhibitor, e.g., it does not bind or inhibit complement pathway activation, e.g., through inhibition of C1, C1r, and C1s. In still another embodiment, a fragment of C1INH specifically binds to or inhibits enodtoxin LPS but does not inhibit contact system activation, e.g., through inhibition of plasma kallikrein, factor XIa, or factor XIIa, for example.

As used herein, the term “active modified C1INH” or “active fragment of C1INH” refers to a modified C1INH polypeptide fragment thereof or a fragment of C1INH which retains the ability to bind to or inhibit endotoxin LPS.

Various aspects of the invention are described in further detail in the following subsections:

### I. ISOLATED NUCLEIC ACID MOLECULES OF THE INVENTION

The coding sequence of the isolated human C1INH cDNA and the predicted amino acid sequence of the human C1INH polypeptide are shown in SEQ ID NOs:1 and 2, respectively, and in FIG. 6. The C1INH sequence is also described in Bock, et al. (1986), Biochemistry 25:4292-4301.

The C1INH nucleic acid molecules of the invention include isolated nucleic acid molecules that encode C1INH proteins, e.g., modified C1INH proteins or fragments thereof, which substantially lack the protease inhibitory activity of naturally occurring C1INH or have reduced or substantially eliminated protease inhibitory activity as compared to naturally occurring C1INH. In one embodiment, the isolated nucleic acid molecules encode a polypeptide comprising amino acids 23-119 of C1INH, or a fragment thereof which is capable of specifically binding or inhibiting LPS.

In another embodiment, the isolated nucleic acid molecules are nucleic acid fragments sufficient for use as hybridization probes to identify C1INH-encoding nucleic acid molecules (e.g., C1INH mRNA) and fragments for use as PCR primers for the amplification or mutation of C1INH nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO:1, or a fragment thereof, can be isolated using standard molecular biology techniques and the sequence information provided herein. Using all or portion of the nucleic acid sequence of SEQ ID NO:1 as a hybridization probe, C1INH nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, or a fragment thereof, can be isolated by the polymerase chain reaction (PCR) using synthetic oligonucleotide primers designed based upon the sequence of SEQ ID NO:1.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to C1INH nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, a complement of the nucleotide sequence shown in SEQ ID NO:1, or a portion of any of these nucleotide sequences. A nucleic acid molecule which is complementary to the nucleotide sequence shown in SEQ ID NO:1, is one which is sufficiently complementary to the nucleotide sequence shown in SEQ ID NO:1 such that it can hybridize to the nucleotide sequence shown in SEQ ID NO:1 thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 91%, 92%, 93%, 94%, 95%, 96%, 97%, 98%, 99% or more identical to the entire length of the nucleotide sequence shown in SEQ ID NO:1 or a portion of any of this nucleotide sequence, e.g., a portion encoding the amino terminal domain of C1INH.

Moreover, the nucleic acid molecules used in the methods of the invention can comprise only a portion of the nucleic acid sequence of SEQ ID NO:1, for example, a fragment which can be used as a probe or primer or a fragment encoding a portion of a C1INH protein, e.g., a biologically active portion of a C1INH protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1 of an anti-sense sequence of SEQ ID NO:1 or of a naturally occurring allelic variant or mutant of SEQ ID NO:1. In one embodiment, a nucleic acid molecule used in the methods of the present invention comprises a nucleotide sequence which is greater than 100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, or more nucleotides in length and hybridizes under stringent hybridization conditions to a nucleic acid molecule of SEQ ID NO:1.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a C1INH protein, such as by measuring a level of a C1INH-encoding nucleic acid in a sample of cells from a subject e.g., detecting C1INH mRNA levels or determining whether a genomic C1INH gene has been mutated or deleted.

The methods of the invention further encompass the use of nucleic acid molecules that differ from the nucleotide sequence shown in SEQ ID NO:1 due to degeneracy of the genetic code and thus encode the same C1INH proteins as those encoded by the nucleotide sequence shown in SEQ ID NO:1. In another embodiment, an isolated nucleic acid molecule included in the methods of the invention has a nucleotide sequence encoding a protein having an amino acid sequence shown in SEQ ID NO:2.

The methods of the invention further include the use of allelic variants of human C1INH, e.g., fuctional and non-functional allelic variants. Functional allelic variants are amino acid sequence variants of the human C1INH protein that maintain a C1INH activity, e.g., the ability to bind LPS, the ability to bind to LPA, the ability to modulate LPS binding to cells, the ability to modulate LPS mediated upregulation of TNF-α or other inflammatory mediators, by cells, or the ability to modulate sepsis or LPS-mediated inflammation in a subject. Functional allelic variants will typically contain only conservative substitution of one or more amino acids of SEQ ID NO:2, or substitution, deletion or insertion of non-critical residues in non-critical regions of the protein.

Non-functional allelic variants are naturally occurring amino acid sequence variants of the human C1INH protein that do not have a C1INH activity, e.g., the ability to bind LPS, the ability to bind to LPA, the ability to modulate LPS binding to cells, the ability to modulate LPS mediated upregulation of TNF-α, or other inflammatory mediators, by cells, or the ability to modulate sepsis or LPS-mediated inflammation in a subject. Non-functional allelic variants will typically contain a non-conservative substitution, deletion, or insertion or premature truncation of the amino acid sequence of SEQ ID NO:2, or a substitution, insertion or deletion in critical residues or critical regions of the protein, e.g., residues or regions necessary for biding to or specifically interacting with LPS.

The methods of the present invention may further use non-human orthologues of the human C1INH protein. Orthologues of the human C1INH protein are proteins that are isolated from non-human organisms and possess the same C1INH activity.

Particular modified C1INH polypeptides which can be made as described herein include modified C1INH polypeptides containing mutations which result in reduced or protease inhibitory activity of the modified C1INH. For example, disruption or cleavage of the serpin reactive domain of C1INH can result in modified C1INH polypeptides which have reduced protease activity but retain the ability to specifically bind to LPS. Furthermore, modified, e.g., truncated C1INH polypeptides which result from the cleavage of amino acids 98-478, or a portion thereof, retain LPS binding activity but have reduced protease inhibitory activity.

The methods of the present invention further include the use of nucleic acid molecules comprising the nucleotide sequence of SEQ ID NO:1 or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of C1INH (e.g., the sequence of SEQ ID NO:2) without altering the biological activity, e.g., specific binding to LPS, whereas an “essential” amino acid residue is required for biological activity. For example, amino acid residues that are conserved among the C1INH proteins of the present invention and other members of the protease inhibitor family are not likely to be amenable to alteration.

Mutations can be introduced into SEQ ID NO:1 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a C1INH protein is preferably replaced with another amino acid residue from the same side chain family. Alternatively, in another embodiment, mutations can be introduced randomly along all or part of a C1INH coding sequence, such as by saturation mutagenesis, and the resultant mutants can be screened for C1INH biological activity to identify mutants that retain activity, e.g., specific binding to LPS. Following mutagenesis of SEQ ID NO:1 the encoded protein can be expressed recombinantly and the activity of the protein can be determined using the assay described herein.

Another aspect of the invention pertains to the use of isolated nucleic acid molecules which are antisense to the nucleotide sequence of SEQ ID NO:1, or fragments thereof. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire C1INH coding strand, or to only a portion thereof. In one embodiment, an antisense nucleic acid molecule is antisense to a “coding region” of the coding strand of a nucleotide sequence encoding a C1INH. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding C1INH. The term “noncoding region” refers to 5′ and 3′ sequences which flank the coding region that are not translated into amino acids (also referred to as 5′ and 3′ untranslated regions).

Given the coding strand sequences encoding C1INH disclosed herein, antisense nucleic acids of the invention can be designed according to the rules of Watson and Crick base pairing. The antisense nucleic acid molecule can be complementary to the entire coding region of C1INH mRNA, but more preferably is an oligonucleotide which is antisense to only a portion of the coding or noncoding region of C1INH mRNA. For example, the antisense oligonucleotide can be complementary to the region surrounding the translation start site of C1INH mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules used in the methods of the invention are typically administered to a subject or generated in situ such that they hybridize with or bind to cellular mRNA and/or genomic DNA encoding a C1INH protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave C1INH mRNA transcripts to thereby inhibit translation of C1INH mRNA. A ribozyme having specificity for a C1INH-encoding nucleic acid can be designed based upon the nucleotide sequence of a C1INH cDNA disclosed herein (i.e., SEQ ID NO:1). For example, a derivative of a Tetrahymena L-19 IVS RNA can be constructed in which the nucleotide sequence of the active site is complementary to the nucleotide sequence to be cleaved in a C1INH-encoding mRNA. See, e.g., Cech et al. U.S. Pat. No. 4,987,071; and Cech et al. U.S. Pat. No. 5,116,742. Alternatively, C1INH mRNA can be used to select a catalytic RNA having a specific ribonuclease activity from a pool of RNA molecules. See, e.g., Bartel, D. and Szostak, J. W. (1993) Science 261:1411-1418.

Alternatively, C1INH gene expression can be inhibited by targeting nucleotide sequences complementary to the regulatory region of the C1INH (e.g., the C1INH promoter and/or enhancers) to form triple helical structures that prevent transcription of the C1INH gene in target cells. See generally, Helene, C. (1991) Anticancer Drug Des. 6(6): 569-84; Helene, C. et al. (1992) Ann. N.Y. Acad. Sci. 660:27-36; and Maher, L. J. (1992) Bioassays 14(12):807-15.

In yet another embodiment, the C1INH nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. (1996) Proc. Natl. Acad. Sci. 93:14670-675.

PNAs of C1INH nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of C1INH nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe et al. (1996) supra).

In another embodiment, PNAs of C1INH can be modified, (e.g., to enhance their stability), by attaching lipophilic or other helper groups to PNA, by the formation of PNA-DNA chimeras, or by the use of liposomes or other techniques of drug delivery known in the art. For example, PNA-DNA chimeras of C1INH nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. et al. (1996) supra). The synthesis of PNA-DNA chimeras can be performed as described in Hyrup B. et al. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

### II. ISOLATED C1INH PROTEINS OF THE INVENTION

The invention includes isolated modified C1INH proteins, and fragments thereof, e.g., C1INH polypeptides which substantially lack the protease inhibitory activity of naturally occurring C1INH or have reduced or substantially eliminated protease inhibitory activity as compared to naturally occurring C1INH. In one embodiment, the invention includes isolated polypeptides comprising amino acids 23-119 of C1INH, or a fragment thereof which is capable of specifically binding or inhibiting LPS. The invention also includes polypeptide fragments suitable for use as immunogens to raise anti-C1INH antibodies. In one embodiment, native C1INH proteins can be isolated from cells or tissue sources by an appropriate purification scheme using standard protein purification techniques. In another embodiment, C1INH proteins are produced by recombinant DNA techniques. Alternative to recombinant expression, a C1INH protein or polypeptide can be synthesized chemically using standard peptide synthesis techniques.

As used herein, a “biologically active portion” of a C1INH protein includes a fragment of a C1INH protein having a C1INH activity, e.g, the ability to bind LPS, the ability to bind to LPA, the ability to modulate LPS binding to cells, the ability to modulate LPS mediated upregulation of TNF-α, or other inflammatory mediators, by cells, or the ability to modulate sepsis or LPS-mediated inflammation in a subject. Biologically active portions of a C1INH protein include peptides comprising amino acid sequences sufficiently identical to or derived from the amino acid sequence of the C1INH protein, e.g., the amino acid sequence shown in SEQ ID NO:2, which include fewer amino acids than the full length C1INH proteins, and exhibit at least one activity of a C1INH protein, e.g., specific binding to LPS. Typically, biologically active portions comprise a domain or motif with at least one activity of the C1INH protein (e.g., the amino-terminal domain of the C1INH protein). A biologically active portion of a C1INH protein can be a polypeptide which is, for example, 5, 10, 15, 20, 25, 30, 35, 40, 45, 50, 55, 60, 65, 70, 75, 80, 85, 90, 95, or 100 or more amino acids in length. Biologically active portions of a C1INH protein can be used as targets for developing agents which modulate a C1INH activity, e.g., binding to LPS, the ability to modulate LPS binding to cells, the ability to modulate LPS mediated upregulation of TNF-α, or other inflammatory mediators, by cells, or the ability to modulate sepsis or LPS-mediated inflammation in a subject.

In a preferred embodiment, the C1INH protein used in the methods of the invention has an amino acid sequence shown in SEQ ID NO:2, or a fragment thereof. In other embodiments, the C1INH protein is substantially identical to SEQ ID NO:2, or a fragment thereof, and retains the functional activity of the protein of SEQ ID NO:2, yet differs in amino acid sequence due to natural allelic variation or mutagenesis, as described in detail in subsection I above. Accordingly, in another embodiment, the C1INH protein used in the methods of the invention is a protein which comprises an amino acid sequence at least about 50%, 55%, 60%, 65%, 70%, 75%, 80%, 85%, 90%, 95%, 96%, 97%, 98%, 99% or more identical to SEQ ID NO:2, or 50%, 55%, 60%, 65%, 70%, 75%, 80%, 85%, 90%, 91%, 92%, 93%, 94%, 95%, 96%, 97%, 98%, 99% or more identical to a fragment of C1INH.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the C1INH amino acid sequence of SEQ ID NO:2 having 500 amino acid residues, at least 75, preferably at least 150, more preferably at least 225, even more preferably at least 300, and even more preferably at least 400 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0U), using a PAM120 weight residue table, a gap length penalty of 12 and a gap penalty of 4.

The methods of the invention may also use C1INH chimeric or fusion proteins. As used herein, a C1INH “chimeric protein” or “fusion protein” comprises a C1INH polypeptide, or a fragment thereof, operatively linked to a non-C1INH polypeptide. A “C1INH polypeptide” refers to a polypeptide having an amino acid sequence corresponding to a C1INH molecule, or a fragment thereof, whereas a “non-C1INH polypeptide” refers to a polypeptide having an amino acid sequence corresponding to a protein which is not substantially homologous to the C1INH protein, or a fragment thereof, e.g., a protein which is different from the C1INH protein and which is derived from the same or a different organism. Within a C1NH fusion protein the C1INH polypeptide, or a fragment thereof, can correspond to all or a portion of a C1INH protein. In a preferred embodiment, a C1INH fusion protein comprises at least one biologically active portion of a C1INH protein, e.g., the amino terminal domain or a fragment thereof. In another preferred embodiment, a C1INH fusion protein comprises at least two biologically active portions of a C1INH protein. Within the fusion protein, the term “operatively linked” is intended to indicate that the C1INH polypeptide and the non-C1INH polypeptide are fused in-frame to each other. The non-C1INH polypeptide can be fused to the N-terminus or C-terminus of the C1INH polypeptide, or a fragment thereof.

For example, in one embodiment, the fusion protein is a GST-C1INH fusion protein in which the C1INH sequences are fused to the C-terminus of the GST sequences. Such fusion proteins can facilitate the purification of recombinant C1INH.

In another embodiment, this fusion protein is a C1INH protein containing a heterologous signal sequence at its N-terminus. In certain host cells (e.g., mammalian host cells), expression and/or secretion of C1INH can be increased through use of a heterologous signal sequence.

The C1INH fusion proteins used in the methods of the invention can be incorporated into pharmaceutical compositions and administered to a subject in vivo. The C1INH fusion proteins can be used to affect the bioavailability of LPS. Moreover, the C1INH-fusion proteins used in the methods of the invention can be used as immunogens to produce anti-C1INH antibodies in a subject, to purify C1INH ligands and in screening assays to identify molecules which inhibit the interaction of C1INH with a C1INH substrate. Preferably, a C1INH chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A C1INH-encoding nucleic acid can be cloned into such an expression vector such that the fusion moiety is linked in-frame to the C1INH protein.

The present invention also pertains to the use of variants of the C1INH proteins which function as C1INH agonists (mimetics). Variants of the C1INH proteins can be generated by mutagenesis, e.g., discrete point mutation or truncation of a C1INH protein. An agonist of the C1INH proteins can retain substantially the same, or a subset, of the biological activities of the naturally occurring form of a C1INH protein, e.g., the ability to bind LPS. Thus, specific biological effects can be elicited by treatment with a variant of limited function.

In one embodiment, variants of a C1INH protein which function as C1INH agonists (mimetics) can be identified by screening combinatorial libraries of mutants, e.g., truncation mutants, of a C1INH protein for C1INH protein agonist activity. In one embodiment, a variegated library of C1INH variants is generated by combinatorial mutagenesis at the nucleic acid level and is encoded by a variegated gene library. A variegated library of C1INH variants can be produced by, for example, enzymatically ligating a mixture of synthetic oligonucleotides into gene sequences such that a degenerate set of potential C1INH sequences is expressible as individual polypeptides, or alternatively, as a set of larger fusion proteins (e.g., for phage display) containing the set of C1INH sequences therein. There are a variety of methods which can be used to produce libraries of potential C1INH variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential C1INH sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a C1INH protein coding sequence can be used to generate a variegated population of C1INH fragments for screening and subsequent selection of variants of a C1INH protein. In one embodiment, a library of coding sequence fragments can be generated by treating a double stranded PCR fragment of a C1INH coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the C1INH protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of C1INH proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify C1INH variants (Arkin and Yourvan (1992) Proc. Natl. Acad. Sci. USA 89:7811-7815; Delgrave et al. (1993) =i Protein Engineering 6(3):327-331).

### III. SCREENING ASSAYS

The invention provides methods (also referred to herein as “screening assays”) for identifying modulators, i.e., candidate or test compounds or agents (e.g., peptides, peptidomimetics, small molecules, ribozymes, or C1INH antisense molecules) which have an inhibitory effect on the activity of a C1INH target ligand, e.g., LPS. Compounds identified using the assays described herein may be useful for treating sepsis.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one aspect, an assay that may be used to identify compounds that modulate C1INH activity, e.g., binding to LPS, the ability to modulate LPS binding to cells, the ability to modulate LPS mediated upregulation of TNF-α, or other inflammatory mediators, by cells, or the ability to modulate sepsis or LPS-mediated inflammation in a subject, is a cell-based assay in which a cell which expresses a C1INH protein or biologically active portion thereof (e.g., the N-terminal region) of the C1INH protein that is necessary for specific binding to LPS, is contacted with a test compound and the ability of the test compound to modulate C1INH activity is determined. Determining the ability of the test compound to modulate C1INH activity can be accomplished by monitoring, for example, LPS binding to macrophages, regulation of LPS-induced TNF-α mRNA or protein, or other inflammatory mediators, or direct binding of modified C1INH, or a fragment thereof, to LPS, as described herein. Other assays known in the art or described herein may be used to determine the ability of a test compound to modulate C1INH activity, e.g., binding to LPS, the ability to modulate LPS binding to cells, the ability to modulate LPS mediated upregulation of TNF-α or other inflammatory mediators by cells, or the ability to modulate sepsis or LPS-mediated inflammation in a subject.

The ability of the test compound to modulate C1INH binding to LPS can also be determined. Determining the ability of the test compound to modulate C1INH binding to LPS can be accomplished, for example, by coupling LPS with a radioisotope or enzymatic label such that binding of LPS to modified C1INH can be determined by detecting the labeled LPS in a complex. Alternatively, modified C1INH could be coupled with a radioisotope or enzymatic label to monitor the ability of a test compound to modulate C1INH binding to LPS in a complex. Determining the ability of the test compound to bind C1INH can be accomplished, for example, by coupling the compound with a radioisotope or enzymatic label such that binding of the compound to C1INH can be determined by detecting the labeled C1INH compound in a complex. For example, C1INH substrates can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to interact with C1INH, e.g., modified C1INH without the labeling of any of the interactants. For example, a microphysiometer can be used to detect the interaction of a compound with C1INH without the labeling of either the compound or the C1INH (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and C1INH, e.g., modified C1INH.

The ability of a C1INH modulator to modulate, e.g., inhibit or increase, C1INH activity can also be determined through screening assays which identify modulators which either increase or decrease binding of C1INH, e.g., modified C1INH, to LPS. In one embodiment, the invention provides for a screening assay involving contacting cells which express a modified C1INH protein or fragment thereof with a test compound and LPS, and measuring the binding of modified C1INH, or a fragment thereof, to LPS, via, e.g., methods described herein.

To determine whether a test compound modulates C1INH expression, in vitro transcriptional assays can be performed. To perform such an assay, the full length promoter and enhancer of modified C1INH can be linked to a reporter gene such as chloramphenicol acetyltransferase (CAT) and introduced into host cells. The same host cells can then be transfected with the test compound. The effect of the test compound can be measured by testing CAT activity and comparing it to CAT activity in cells which do not contain the test compound. An increase or decrease in CAT activity indicates a modulation of modified C1INH expression and is, therefore, an indicator of the ability of the test compound to bind to LPS.

In yet another embodiment, an assay of the present invention is a cell-free assay in which a C1INH protein or biologically active portion thereof (e.g., the N-terminal domain of the C1INH, e.g., modified C1INH protein that is involved in the binding to and inhibition of LPS by C1INH, e.g., modified C1INH) is contacted with a test compound and the ability of the test compound to bind to or to modulate (e.g., stimulate or inhibit) the activity of the C1INH, e.g., modified C1INH protein or biologically active portion thereof is determined. Preferred biologically active portions of the modified C1INH proteins to be used in assays of the present invention include fragments which are capable of specifically binding LPS, e.g., fragments comprising amino acids 23-119 of C1INH, or a fragment thereof. Determining the ability of the C1INH, e.g., modified C1INH protein to bind to a test compound can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In more than one embodiment of the above assay methods of the present invention, it may be desirable to immobilize either C1INH, e.g., modified C1INH or LPS to facilitate separation of complexed from uncomplexed forms of one or both of the proteins, as well as to accommodate automation of the assay. Binding of a test compound to a modified C1INH protein, or interaction of a modified C1INH protein with LPS in the presence and absence of a test compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/C1INH fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or C1INH protein, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of C1INH binding or activity determined using standard techniques.

Other techniques for immobilizing proteins on matrices can also be used in the screening assays of the invention. For example, either a modified C1INH protein or LPS can be immobilized utilizing conjugation of biotin and streptavidin. Biotinylated C1INH protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with modified C1INH protein or target molecules but which do not interfere with binding of the modified C1INH protein to its target molecule can be derivatized to the wells of the plate, and unbound target or C1INH, e.g., modified C1INH protein is trapped in the wells by antibody conjugation. Methods for detecting such complexes, in addition to those described above for the GST-immobilized complexes, include immunodetection of complexes using antibodies reactive with the modified C1INH protein or LPS, as well as enzyme-linked assays which rely on detecting an enzymatic activity associated with the C1INH protein LPS.

In yet another aspect of the invention, the C1INH, e.g., modified C1INH protein or fragments thereof (e.g., the N-terminal domain of the C1INH, e.g., modified C1INH protein that is involved in the binding to and inhibition of LPS) can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with C1INH, e.g., modified C1INH (“C1INH-binding proteins” or “C1INH-bp) and are involved in C1INH activity. Such modified C1INH-binding proteins are also likely to be involved in the propagation of signals by the modified C1INH proteins as, for example, downstream elements of a C1INH-mediated signaling pathway. Alternatively, such C1INH-binding proteins are likely to be C1INH inhibitors.

The two-hybrid system is based on the modular nature of most transcription factors, which consist of separable DNA-binding and activation domains. Briefly, the assay utilizes two different DNA constructs. In one construct, the gene that codes for a C1INH protein, e.g., a modified C1INH protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a C1INH, e.g., modified C1INH-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the C1INH, e.g., modified C1INH protein.

In another aspect, the invention pertains to a combination of two or more of the assays described herein. For example, a modulating agent can be identified using a cell-based or a cell-free assay, and the ability of the agent to modulate the activity of a C1INH protein can be confirmed in vivo, e.g., in an animal such as an animal model for sepsis. Examples of animals that can be used include animals, e.g., mice, rabbits, or baboons, which have been administered a gram-negative organism, e.g., E. coli, in order to induce sepsis in the animal, as described in, for example, Jansen, et al. (1998) J. of Immunol 160:475-484 and Scherer, et al. (1996) Sem in Thromb and Hemostasis 22:357-366).

Moreover, a C1INH modulator identified as described herein (e.g., an antisense C1INH nucleic acid molecule, a C1INH-specific antibody, or a small molecule) can be used in an animal model to determine the efficacy, toxicity, or side effects of treatment with such a modulator. Alternatively, a C1INH modulator identified as described herein can be used in an animal model to determine the mechanism of action of such a modulator.

### IV. PREDICTIVE MEDICINE

The present invention also pertains to the field of predictive medicine in which diagnostic assays and prognostic assays are used for prognostic (predictive) purposes to thereby treat an individual prophylactically. For example, the methods and compositions of the invention can be used to prevent sepsis in high-risk subjects (e.g., immunocompropmised subjects such as surgical and other hospitalized patients, patients receiving cancer therapy, low birth weight infants, and burn and trauma victims). Accordingly, one aspect of the present invention relates to diagnostic assays using C1INH, or a fragment thereof, for determining whether an individual is afflicted with sepsis. The invention also provides for prognostic (or predictive) assays for determining whether an individual is at risk of developing sepsis.

To determine whether a subject is afflicted with sepsis, a biological sample may be obtained from a subject and the biological sample may be contacted with a compound or an agent capable of detecting LPS in the biological sample, e.g., modified C1INH or a fragment thereof capable of binding LPS. Binding of modified C1INH to LPS may be determined by an assay described herein to directly detect binding, or by any assay known in the art. Moreover, binding by modified C1INH to LPS may also be determined by assays which monitor the levels of inflammatory mediators, e.g., TNF-α, the release of which is modulated by the presence of LPS. The term “biological sample” is intended to include tissues, cells, and biological fluids isolated from a subject, as well as tissues, cells, and fluids present within a subject. That is, the detection method of the invention can be used to detect LPS in a biological sample in vitro as well as in vivo.

In another embodiment, the methods further involve obtaining a control biological sample from a control subject, contacting the control sample with a modified C1INH polypeptide, such that the presence of LPS is detected in the biological sample, and comparing the presence of LPS in the control sample with the presence of LPS in the test sample.

### V. METHODS OF TREATMENT OF SUBJECTS SUFFERING FROM SEPSIS

The present invention provides for both prophylactic and therapeutic methods of treating a subject, e.g., a human, at risk of (or susceptible to) sepsis. With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring an subject's prophylactic or therapeutic treatment with either the modified C1INH molecules of the present invention or C1INH modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing sepsis in a subject by administering to the subject modified C1INH, or a fragment thereof which specifically binds LPS. Subjects at risk for sepsis can be identified by, for example, any or a combination of the diagnostic or prognostic assays described herein. Administration of a prophylactic agent can occur prior to the manifestation of symptoms characteristic of sepsis, e.g., prior to a procedure, e.g., a surgical procedure, which places a subject at risk for sepsis, such that sepsis is prevented or, alternatively, delayed in its progression.

B. Therapeutic Methods

Another aspect of the invention pertains to methods for treating a subject suffering from sepsis. These methods involve administering to a subject a modified C1INH protein, or fragment thereof, or a C1INH mimetic, e.g., a small molecule, as therapy for sepsis. Administration of modified C1INH, or a fragment thereof, to a subject for the treatment or prevention of sepsis may be alone or in combination with other agents known to aid in the treatment or prevention of sepsis, e.g., antibiotics, anti-TNF-α antibodies, and/or anti-LPS antibodies, which may be produced as described in U.S. Pat. No. 6,315,999, the contents of which are incorporated herein by reference). In another embodiment, when therapeutically beneficial, modified C1INH, or a fragment thereof, may be administered in combination with any agent which acts as a protease inhibitor to inhibit the complement system, e.g., through inhibition of C1, C1s, or C1r, and/or any agent which inhibits contact system activation, e.g., through inhibition of plasma kallikrein, factor XIa, or factor XIIa, for example. Administration of modified C1INH and another agent may be serially or as a mixture.

C1INH, e.g., modified C1INH, or a fragment thereof, can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating modified C1INH or a fragment of a C1INH protein in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

C1INH, e.g., modified C1INH or a fragment thereof can also be prepared in the form of suppositories (e.g., with conventional suppository bases such as cocoa butter and other glycerides) or retention enemas for rectal delivery.

In one embodiment, C1INH, e.g., modified C1INH or a fragment thereof is prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates C1INH activity and the particular therapeutic effect to be achieved, and the limitations inherent in the art of compounding such an agent for the treatment of subjects.

Toxicity and therapeutic efficacy of C1INH, e.g., modified C1INH or a fragment thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such modified C1INH or a fragment thereof lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which mimic C1INH LPS binding activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNIU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

C. Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer C1INH or a fragment thereof, as well as tailoring the dosage and/or therapeutic regimen of treatment with C1INH, or a fragment thereof.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known, all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a modified C1INH molecule or C1INH modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from sepsis with a modified C1INH polypeptide.

### VI. RECOMBINANT EXPRESSION VECTORS AND HOST CELLS USED IN THE METHODS OF THE INVENTION

The methods of the invention (e.g., the screening assays described herein) include the use of vectors, preferably expression vectors, containing a nucleic acid encoding a modified C1INH protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., C1INH proteins, mutant forms of C1INH proteins, fragments of C1INH proteins, fusion proteins, and the like).

The recombinant expression vectors to be used in the methods of the invention can be designed for expression of C1INH proteins in prokaryotic or eukaryotic cells. For example, C1INH proteins can be expressed in bacterial cells, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in C1INH activity assays, (e.g., direct assays or competitive assays described in detail herein), or to generate antibodies specific for C1INH proteins. In a preferred embodiment, a C1INH fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to C1INH mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a C1INH nucleic acid molecule of the invention is introduced, e.g., a C1INH nucleic acid molecule within a recombinant expression vector or a C1INH nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a C1INH protein can be expressed in bacterial cells, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a C1INH protein. Accordingly, the invention further provides methods for producing a C1INH protein using the host cells of the invention. In one embodiment, the method comprises culturing the host cell of the invention (into which a recombinant expression vector encoding a C1INH protein has been introduced) in a suitable medium such that a C1INH protein is produced. In another embodiment, the method further comprises isolating a C1INH protein from the medium or the host cell.

This invention is further illustrated by the following examples which should not be construed as limiting. The contents of all references, patents and published patent applications cited throughout this application, as well as the Figures and Sequence Listing, are incorporated herein by reference.

### EXAMPLES

### Example 1

### C1Inhibitor Interacts Directly with Gram-Negative Endotoxin Lipopolysaccharide (LPS)

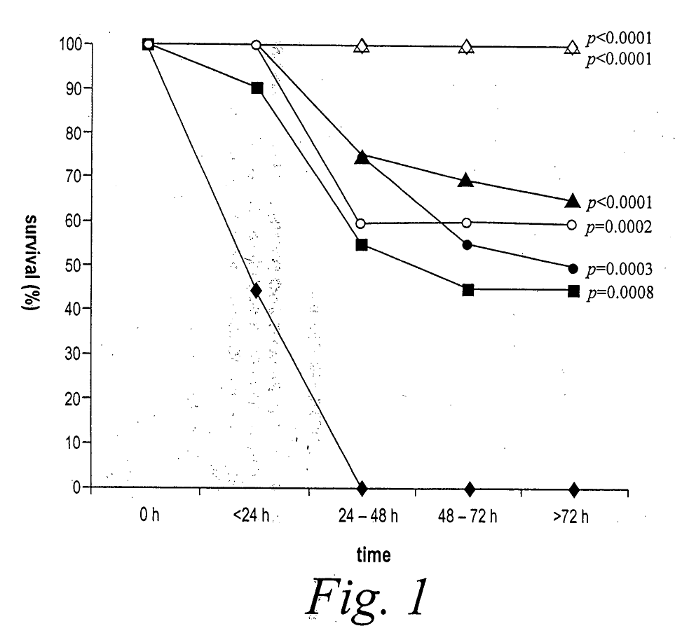

The ability of both native, active C1INH and reactive center cleaved C1INH, derived from C1INH by trypsin treatment, was analyzed to improve the survival of Gram-negative endotoxin LPS-induced endotoxemia in mice. The lowest dose of LPS (20 mg/kg) that resulted in 100% mortality of C57BL/6J mice within 48 hours was selected. A single dose of C1INH (200 μg/per mouse) improved survival at 72 hours to 45% and 50%, when administered via the intraperitoneal (i.p.) and intravenous (i.v.) routes, respectively (FIG. 1). In addition, a mixture of C1INH with LPS administered i.p. increased survival to 65%. Reactive center cleaved C1INH also protected mice, with an increase in survival to 60% after i.v. administration and to 100% when given i.p. after mixing with LPS. These data confirm previous observations that C1INH protects mice from mortality secondary to endotoxin LPS. Furthermore, the observation that mixing C1INH with LPS prior to i.p. administration is more effective than separate i.v. or i.p. administration of C1INH, suggests that C1INH interacts specifically with LPS. These data also indicate that C1INH, in addition to its function as a serine protease inhibitor, acts to prevent the adverse biologic effects of LPS via a mechanism unrelated to its protease inhibitory activity.

LPS binding to macrophages via LBP transfers LPS to CD14 which initiates intracellular signaling and results in production of inflammatory mediators by binding to toll-like receptors (Fenton, M. J. & Golenbock, D. T. (1998) J Leukocyte Biol 64:25-32; Poltorak, A. et al. (1998) Science 282:2085-2088; Schumann, R. R. & Latz, E. (2000) Chem Immunol 74:42-60; Kitchens, R. L. (2000) Chem Immunol 74). Whether C1INH blocks LPS binding to macrophages was tested. C1INH at concentrations of 37.5-150 μg/ml, which are within the physiological concentration range in human plasma (Nuijens, J. H. et al. (1989) J Clin Invest 84:443-450), in the presence of 10% FBS completely blocked the binding of LPS (175 ng/ml) to the murine macrophage cell line, RAW 264.7 (FIG. 2A). The fluorescence intensity was also decreased when macrophages were pretreated with C1INH (150 μg/ml) for 15 min at 37° C., following which the C1INH was removed and LPS was added to the cells. However, the effect was only observed at a lower concentration of LPS (40 ng/ml), and was not apparent when 175 ng/ml LPS was used (FIG. 2B). This suggests that C1INH interacts with endotoxin rather than with a cellular receptor. Anti-human C1INH antibody completely abrogated the effect of C1INH on LPS binding to macrophages, whereas BSA (100 μg/ml) did not interfere (FIG. 2C). The binding of LPS to macrophages by fluorescence microscopy was also tested. RAW 264.7 cells were cultured on microscope slides (Fisher Scientific, Pittsburgh, Pa.) in the presence of FITC-conjugated LPS that had been incubated with or without C1INH. The fluorescent signals were decreased at a concentration of 50 μg/ml C1INH and completely eliminated at 100-150 μg/ml C1INH (FIG. 2D).

LPS activates macrophages to produce and release potent inflammatory mediators, of which TNF-α is very important for the development of endotoxin shock (Nagaoka, I. et al. (2001) J Immunol 167:3329-3338). Expression of LPS-induced TNF-α mRNA in RAW 264.7 cells by RT-PCR was detected. LPS-mediated upregulation of TNF-α mRNA was completely suppressed following treatment with 150 μg /ml C1INH (FIGS. 2E & F). Therefore, C1INH mediated inhibition of LPS binding to macrophages suppresses LPS-induced TNF-α mRNA.

Cleavage of the serpin reactive center loop by either target or non-target proteases results in a structural rearrangement with complete insertion of the loop into the five-stranded β sheet A. In the case of non-target proteases, the active protease is released, while target proteases remain covalently bonded to the serpin via the P1 residue (Loebermann, H., et al. (1984) J Mol Biol 177:531-57; Eldering, E., et al. (1993) J Clin Invest 92:1035-1043; Djie, M. Z., et al. (1996) Biochemistry 35:11461-11469; Huntington, J. A., et al. (2000) Nature 407:923-926). To investigate whether the molecular specificity within the C1INH reactive center loop is related to inhibition of LPS binding to macrophages, the reactive center loop of C1INH was cleaved (150, 300, 600 μg/ml) at P1-P1′ with trypsin. Both C1INH-C1s complexes (FIG. 3A) and reactive center loop cleaved C1INH (FIG. 3B) retained the ability to block LPS binding to macrophages. Therefore, an intact reactive center loop is not required for this inhibition.

To investigate the possibility that C1INH interacts specifically with Gram-negative endotoxin LPS, LPS was immobilized in microtiter wells at a variety of concentrations and measured the amount of C1INH binding in the presence or absence of BSA (100 μg/ml) or IgG (20 μg/ml). The binding of 150 μg/ml C1INH was maximal to 175 ng/ml LPS and neither BSA nor IgG interfered with this binding (FIG. 4A). FBS and the human LPS-binding LBP peptides reduced C1INH binding to LPS by approximately 80% and 75%, respectively (FIGS. 4B & C). Binding of the negatively charged LPS to proteins resulted in a characteristic anodal shift in the mobility of the protein on native polyacrylamide gel electrophoresis (PAGE)(Hailmen, E. et al. (1994) J Exp Med 179, 269-277; Viriyakosol, S., et al. (2001) J Biol Chem 276:38044-38051). The interaction of C1INH with LPS by native PAGE was analyzed and immunoblotted with anti-human C1INH antibody. C1INH also demonstrated an anodal shift, which increased with increasing amounts of LPS (FIG. 4D). C1INH incubated with LPS for 30 min at 37° C. prior to the addition of C1s had no effect on either the rate or extent of complex formation with C1s in comparison with C1INH incubated with C1s in the absence of endotoxin (FIG. 4E). Therefore, Gram-negative endotoxin LPS neither enhances nor suppresses the ability of C1INH to complex with target protease.

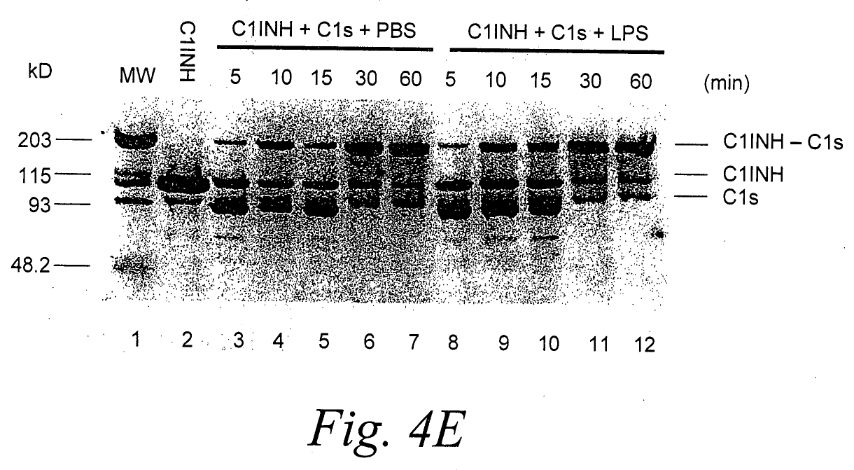

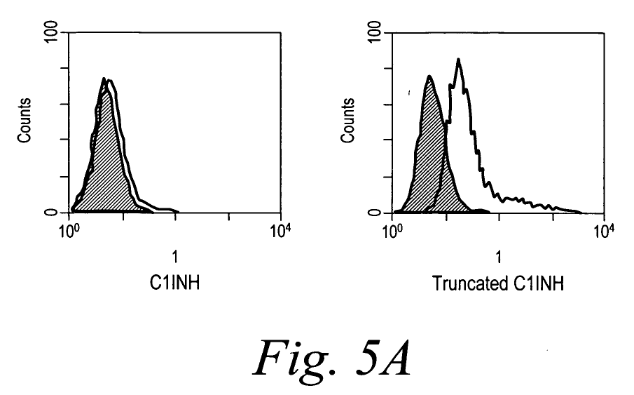

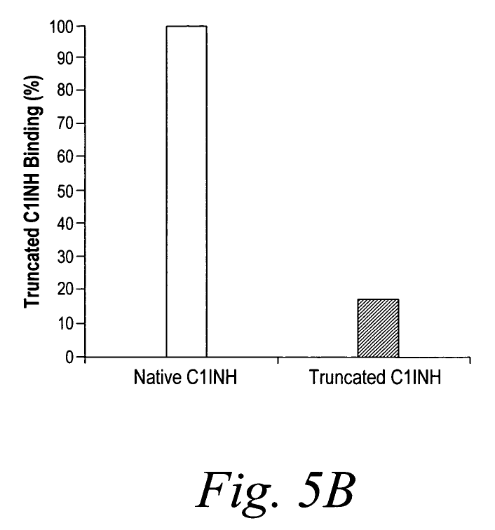

C1INH has an amino terminal heavily glycosylated mucin-like domain (amino acids 23 to 142) that contains 7 repeats of the tetrapeptide sequence Glx-Pro-Thr-Thr, or variants thereof. This domain does not appear to influence complex formation with target proteases. The serpin domain consists of residues 142 through the C-terminus (Bock, S. C. et al. (1986) Biochemistry 25:4292-4301; Coutinho, M., et al. (1994) J Immunol 153:3648-3654). The functional significance of the N-terminal extension of C1INH is unknown, although several possibilities have been suggested (Coutinho, M., et al. (1994) J Immunol 153:3648-3654; Perkins, S. (1990) FEBS Lett 271:89-92). The hypothesis that the C1INH N-terminal domain is responsible for the interaction with LPS was tested. A truncated C1INH molecule consisting of amino acid residues 120 to 500 (Coutinho, M., et al. (1994) J Immunol 153:3648-3654), did not block LPS binding to macrophages (FIG. 5A), but the full-length C1INH protein in the presence of the truncated protein was able to block binding (data not shown). In addition, the binding of truncated C1INH to LPS was reduced by 85% compared with full-length C1INH (FIG. 5B). The amino terminal domain, therefore, is required for inhibition of LPS-mediated macrophage activation.

Although protease inhibition, the major biological function of C1INH, requires an intact reactive center loop, it was found that cleaved inactive C1INH protected mice from lethal endotoxemia and blocked the binding of LPS to macrophages. This protection is a result of a direct interaction with LPS, which is mediated via the heavily glycosylated amino terminal domain. It, therefore, appears likely that C1INH contributes to protection from Gram negative endotoxin shock via three mechanisms: inhibition of excessive complement activation which would limit the amount of C5a generated, inhibition of contact system activation which would limit the amount of activated plasma kallikrein, factor XIIa and bradykinin generated, and by direct inhibition of endotoxin binding to macrophages which thereby suppresses macrophage activation.

Methods

Mouse Endotoxemia Model

C57BL/6J mice (male and female, 6-8 weeks, 18-22 g)(Charles River Laboratories, Wilmington, Mass.) were injected i.p with a lethal dose of LPS (20 mg/kg) following treatment i.p or i.v with C1INH (200 μg/mouse) or reactive center cleaved C1INH (200 μg/per mouse). In other experiments, mice were injected i.p with a mixture of LPS (20 mg/kg) and C1INH (200 μg/per mouse) or cleaved C1INH (200 μg/per mouse). Control mice were injected with LPS (i.p.) or C1INH (i.v.) alone. Animals were cared for under standard conditions and were monitored for 5 days. Each of the treated groups of mice were compared with the group that received LPS alone using the Logrank Test (GraphPad Prism version 3.0, GraphPad Software, Inc., San Diego, Calif.). All experiments were performed in compliance with relevant laws and institutional guidelines, and were approved by the Center for Blood Research Animal Care and Use Committee.

Flow Cytometry Analysis (FACS)

RAW 264.7 murine macrophage cells were incubated with FITC-conjugated LPS from Salmonella typhimurium (Sigma Chemical Company, St. Louis, Mo.) in the presence of either native C1INH (Advanced Research Technologies, San Diego, Calif.), C1INH-C1s complexes, reactive center cleaved C1INH, or a recombinant C1INH protein with deletion of the amino terminal 98 amino acids (expressed in Hi5 insect cells) (Coutinho, M., et al. (1994) J Immunol 153:3648-3654), in DMEM containing 10% FBS for 15 min at 37° C. C1INH-C1s complexes were prepared by incubation of equimolar quantities of C1INH and C1s (Advanced Research Technologies) for 60 min at 37° C. and reactive center cleaved C1INH was generated by incubation of native C1INH with trypsin attached to cross-linked agarose (Sigma Chemical Company) for 15 min at 37° C. (Zahedi, R., (1997) J Immunol 159:983-988; Zahedi, R., et al. (2001) J Immunol 167, 1500-1506). In other experiments, the macrophages were incubated with FITC-conjugated LPS in addition to C1INH which was pretreated with rabbit anti-human C1INH antibody (Dako, Denmark) for 1 hour at 37° C. Cells were fixed with FACS solution after washing with PBS three times and were analyzed on a FACS Calibur (Becton Dickinson, San Jose, Calif.) using CellQuest software.

Fluorescence Microscopy

Binding of FITC-conjugated LPS to macrophages was done as described previously (Hailmen, E. et al. (1994) J Exp Med 179:269-277; Viriyakosol, S., et al. (2001) J Biol Chem 276:38044-38051). Fluorescence localization was evaluated and measured by Axiophot fluorescence microscopy (Zeiss, Oberlochen, Germany) with a green fluorescence filter set.

RT-PCR

Total cytoplasmic RNA was isolated from the RAW 264.7 macrophages (Chomcyzynski, P. & Sacchi, N. (1987) Anal Biochem 162:156-159), and reverse transcribed to cDNA using M-MuLV reverse transcriptase (BioLabs, Beverly, Mass.) with Oligo (dT)₂₀ primers (Invitrogen) for 1 hour at 37° C. PCR primers were designed to generate TNF-α and β-actin fragments with lengths of 200 bp. PCR products were analyzed on 1.2% (w/v) agarose gels containing 0.5 μg/ml ethidium bromide and visualized under UV light. The band density was analyzed and quantified using ImageQuant software (Molecular Dynamics, Sunnyvale, Calif.).

ELISA

Plates (COSTAR, Coming Incorporated, Coming, N.J.) were coated with Salmonella typhimurium LPS (Sigma Chemical Company) or C1INH at 4° C. overnight. Control plates were incubated with BSA (100 μg/ml), IgG (20 μg/ml) or PBS in the absence of C1INH. C1INH (150 μg/ml) was incubated with LPS coated plates for 1 hour at room temperature in the presence or absence of FBS (10-100 μl) or human LBP peptides (Cell Sciences, Inc., Norwood, Mass.)(5-40 ng/ml). Rabbit anti-human C1INH antibody (1:1000) was incubated for 1 hour at room temperature following which plates were incubated with ImmunoPure goat anti-rabbit IgG (H+L) conjugated with horseradish peroxidase (1:100,000)(Pierce, Rockford, Ill.). After washing with PBS, o-phenylenediamine dihydrochloride (Sigma) substrate was added and the color reactions were developed for 5 minutes at room temperature and terminated with 3 N HCl. Absorbance was measured at 490 nm using the Revelation Microsoft in an MRX microplate reader (DYNEX Technologies, Chontilly, Va.).

PAGE and Western Blotting

Native PAGE of C1INH (10 μg/ml) incubated with various concentrations of LPS (37° C., 30 minutes in PBS) was performed as previously described (Hailmen, E. et al. Lipopolysaccharide (LPS)-binding protein accelerated the binding of LPS to CD14. J Exp Med 179, 269-277 (1994)). Proteins were electrophoretically transferred to nitrocellulose membranes (Invitrogen), blocked with 5% fat-free milk (BIO-RAD) in PBST (1× PBS, pH 7.4, plus 0.05% Tween) at 4° C. overnight, incubated with rabbit anti-human C1INH antibody (1:1000) in 5% fat-free milk for 2 hour at room temperature, washed with 1× PBS for 20 minutes, and incubated with a 1:10,000 dilution of ImmunoPure goat anti-rabbit IgG (H+L) conjugated with horseradish peroxidase for 2 hour at room temperature. After washing with PBS for 20 minutes, development was performed using a SuperSignal Chemiluminescent Substrate kit (PIERCE). For SDS-PAGE, C1INH was incubated with C1s after the addition of LPS (175 ng/ml) for 5, 10, 30, and 60 minutes at 37° C. Loading buffer was added, and the mixture was subjected to electrophoresis using a 10% SDS-Tris-Glycine polyacrylamide gel (Invitrogen). The gel was stained with Coomassie blue and dried (1 hour, 80° C.).

### Example 2

### N-Linked Glycoslation is Required for Cl Inhibitor-Mediated Protection from Endotoxin Shock in Mice

Methods

Reagents.

C1INH and C1s were from Advanced Research Technologies (San Diego, Calif.). Moloney marine leukemia virus (M-MuLV) reverse transcriptase, N-glycosidase F (from Flavobacterium meningosepticum), and neuraminidase (from Arthrobacter ureafaciens) were purchased from New England BioLabs (Beverly, Mass.). O-glycosidase (from Diplococcus pneumoniae) was from Roche Diagnostics GmbH (Mannheim, Germany), o-phenylenediamine dihydrochloride substrates, LPS and fluorescein isothiocyanate (FITC)-conjugated LPS from Salmonella typhimurium were obtained from Sigma Chemical Co. (St. Louis, Mo.), as were Salmonella typhimurium LPS derived monophosphoryl lipid A (mLPA), diphosphoryl lipid A (dLPA) and detoxified lipopolysaccharide (dLPS). Rabbit anti-human C1INH antibody was purchased from DAKO (Glostrup, Denmark). Goat anti-rabbit IgG (H+L) conjugated with horseradish peroxidase was purchased from Pierce Biotechnology (Rockford, Ill.).

Enzymatic Deglycosylation of C1INH.

Deglycosylation was performed as described (da Silva Correia et al. (2002) J Biol. Chem. 277:1845-1854). N-linked carbohydrate was removed from plasma-derived C1INH by treatment with 50 units/ml N-glycosidase F for 2 hours at 37° C. O-linked carbohydrate was removed by incubation with 0.1 unit/ml O-glycosidase and 0.1 unit/ml neuraminidase overnight at 37° C. To remove both N— and O-linked carbohydrates, C1INH (10 μg/ml) was treated with 50 units/ml of N-glycosidase F, 0.1 unit/ml of O-glycosidase and 0.1 unit/ml, neuraminidase overnight at 37° C.

Mouse Endotoxin Shock Model.

C57BL/6J mice (male, 6-8 weeks, 18-22 g) (Charles River Laboratories, Wilmington, Mass.) were injected i.p with a lethal dose (20 mg/kg) of Salmonella typhimurium LPS mixed with either normal intact, N-deglycosylated, O-deglycosylated or N— and O-deglycosylated C1INH (200 μg/mouse). Control mice were injected (i.p.) with LPS (20 mg/kg) alone or in a mixture of LPS (20 mg/kg) with the buffer used for deglycosylation. Mice were monitored for 5 days. Each of the treated groups was compared with the control group that received LPS alone using the Logrank Test (GraphPad Prism version 3.0, GraphPad Software, Inc., San Diego, Calif.).

Cell Culture.

The murine RAW 264.7 macrophage cell line (ATCC, Manassas, Va.) was cultured in Dulbecco's Minimum Essential Medium (DMEM)(ATCC, Manassas, Va.) supplemented with 10% fetal bovine serum (FBS) (Invitrogen, Carlsbad, Calif.) at 37° C. in 5% CC>2. Confluent macrophages were detached by washing with phosphate buffered saline (PBS)(pH 7.4).

FACS.

The murine RAW 264.7 macrophages were incubated with FITC-conjugated LPS (175 ng/ml) in the absence or the presence of the various deglycosylated C1INH (150 μg/ml) preparations in DMEM containing 10% FBS for 15 minutes at 37° C. In other experiments, human peripheral venous blood from a normal volunteer was collected in EDTA (1 mg/ml whole blood). Aliquots of the whole blood were treated with LPS at a final concentration of 175 ng/ml in the absence and the presence of added N-deglycosylated C1INH (150 μg/ml), O-deglycosylated C1INH (150 μg/ml), or N— and O-deglycosylated C1INH (150 μg/ml) for 15 minutes at 37° C. Cells were fixed with fluorescence activated cell sorter (FACS) solution after washing with PBS three times. The binding of FITC-conjugated LPS was analyzed on FACS Calibur (Becton Dickinson, San Jose, Calif.) using CellQuest software.

SDS-PAGE.

C1INH was incubated with C1s (ratio=1:1) for 20 minutes at 37° C. after the addition of mLPA, dLPA, or dLPS from Salmonella typhimurium LPS. Reactions were subjected to electrophoresis using a 10% SDS-Tris-Glycine polyacrylamide gel (Invitrogen, Carlsbad, Calif.). The gels were stained with Coomassie blue and then dried for 1 hour at 80° C.

RT-PCR.

Total RNA was isolated from the RAW 264.7 macrophages induced with LPS (175 ng/ml) in the presence or absence of N-deglycosylated C1INH, O-deglycosylated C1INH, or N— and O-deglycosylated C1INH, and reverse transcribed using M-MuLV reverse transcriptase with Oligo dT₂₀ primers (Invitrogen, Carlsbad, Calif.)(1 hour, 37° C.). PCR primers were designed to generate mouse TNF-α and β-actin fragments, each with lengths of 200 bp (TNF-α, sense: 5′-ATGAGCACAGAAAGCATGATCC-3′ (SEQ ID NO:3) and antisense: 5′-GAGGCCATTTGGGAACTTCTC-3 (SEQ ID NO:4); β-actin, sense: 5′-TGGATGACGATATCGCTGC-3′ (SEQ ID NO:5) and antisense: 5′-AGGGTCAGGATACCTCTCTT-3′) (SEQ ID NO:6). PCR products were analyzed on 1.2% (w/v) agarose gels containing 0.5 μg/ml ethidium bromide and visualized under UV light. Band density was analyzed and quantified using ImageQuant software (Molecular Dynamics, Sunnyvale, Calif.). In addition, human peripheral venous blood from a normal volunteer was collected in EDTA (1 mg/ml whole blood). Aliquots of the whole blood were treated with LPS at a final concentration of 175 ng/ml in the absence and the presence of added C1INH (5-150 μg/ml) for 15 minutes at 37° C. In other experiments, total RNA was isolated from the blood leukocytes and was reverse transcribed using M-MuLV reverse transcriptase with Oligo dT₂₀ primers. PCR primers were designed for human TNF-α (sense: 5′-ATGAGCACTGAAAGCATGATCCGGGACGTG-3′ (SEQ ID NO:7) and antisense: 5′-AGGTCCCTGGGGAACTCTTCCCTCTG-3) (SEQ ID NO:8) and human β-actin (sense: 5′-ATGGATGATGATATCGCCGCGCTCGTCGTC-3′ (SEQ ID NO:9) and antisense: 5′-AGGGTGAGGATGCCTCTCTTGCTCTG-3′ (SEQ ID NO:10)).

ELISA.

Plates (Polyvinyl Chloride, 96 of U-Bottom well) (BECTON DICKINSON, Frankin Lakes, N.J.) were coated with mLPA, dLPA, dLPS, and LPS at room temperature overnight. C1INH (150 W/ml) was incubated with mLPA, dLPA, dLPS, and LPS coated plates for 1 hour at room temperature, respectively. In other experiments, deglycosylated C1INH (150 (μg/ml) was treated for 2 hours at 37° C. or overnight at 37 ° C. and was stopped by chilling and then incubated with LPS (175 ng/ml) coated plates for 1 hour at room temperature. Rabbit anti-human C1INH antibody (1:1000) was incubated for 1 hour at room temperature. After washing, plates were incubated with ImmunoPure goat anti-rabbit IgG (H+L) conjugated with horseradish peroxidase (1:100,000). Color reactions were developed for 3 minutes at room temperature and reactions were terminated with 3 N HCL. The absorbance of each well was measured at 490 nm using Revelation Microsoft in an MRX microplate reader (DYNEX Technologies, Chontilly, Va.). Standard samples were detected with the different concentrations of human CIINH binding to rabbit anti-human CIINH antibody.

Results

Analysis of Deglycosylated C1INH.

Treatment of plasma-derived intact C1INH with neuraminidase reduced the apparent molecular weight, as judged by SDS-PAGE, from 106 kDa to 96 kDa (FIG. 7). Removal of N— and O-linked carbohydrate resulted in decreases to 89 kDa and 83 kDa, respectively, while the combination of N— and O-glycosidase treatment reduced the apparent molecular weight to 75 kDa. These apparent size differentials are consistent with previously published data (see Reboul (1987) Biochem J 24:117-121).

Removal of N-Linked Carbohydrate Abrogates the Ability of C1INH to Protect Mice from Lethal Endotoxin Shock.

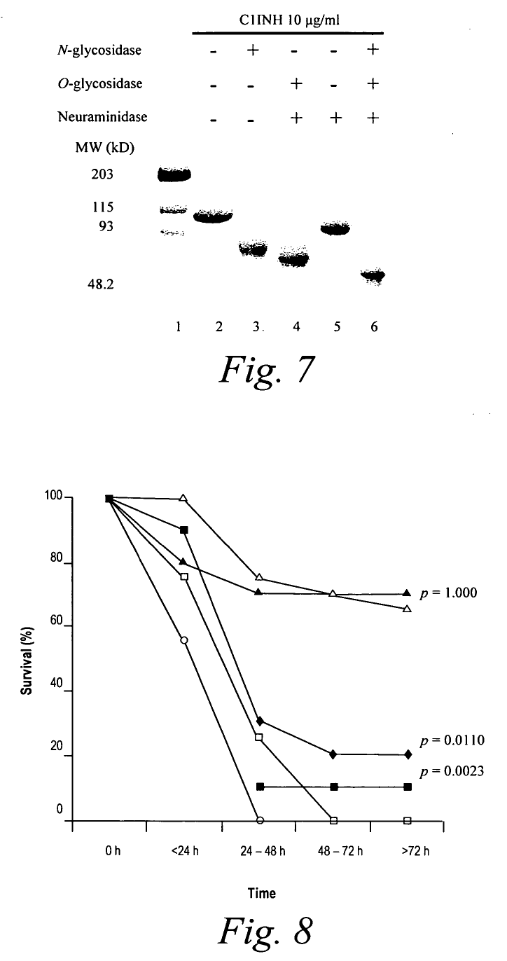

C1INH prevented the adverse biologic effects of LPS via a mechanism unrelated to protease inhibition and this protection appeared to be secondary to a direct interaction with endotoxin. Deletion of the amino terminal 119 amino acid residues abrogated the ability of C1INH to bind to LPS in vitro (Liu et al (2003) J. Immunol. 171:2594-2601). However, the available quantities of this recombinant truncated C1INH are insufficient for testing in animals. Using the lowest dose of LPS (20 mg/kg) that resulted in 100% mortality of C57BL/6J mice within 48 hours, it was demonstrated that a single dose of C1INH (200 μg) improved survival to 45% and 50% when administered via the intraperitoneal (i.p.) and intravenous (i.v.) routes, respectively. A mixture of LPS (20 mg/kg) and C1INH (200 jig/per mouse) i.p. increased survival to 65% (Liu, D., et al. (2003) J Immunol. 171:2594-2601). In order to evaluate the role of carbohydrate in C1INH-mediated protection from endotoxin shock, mice were injected i.p. with LPS mixed with either native C1INH, N-deglycosylated C1INH, O-deglycosylated C1INH or N— and O-deglycosylated C1INH (200 μg/mouse). Treatment with native C1INH and with O-deglycosylated C1INH resulted in 65% and 70% survival, respectively (FIG. 8). N-deglycosylated and N-+O-deglycosylated C1INH, on the other hand, resulted in survival rates of only 20% and 10%, respectively, at 72 hours. These data indicate that the N-linked glycosylation of C1INH is essential to protect mice from lethal LPS-induced endotoxin shock.

C1INH Lacking N-Glycosylation Failed to Suppress LPS Binding to the Murine Macrophage Cell Line, RAW 264.7, and to Human Blood Cells.

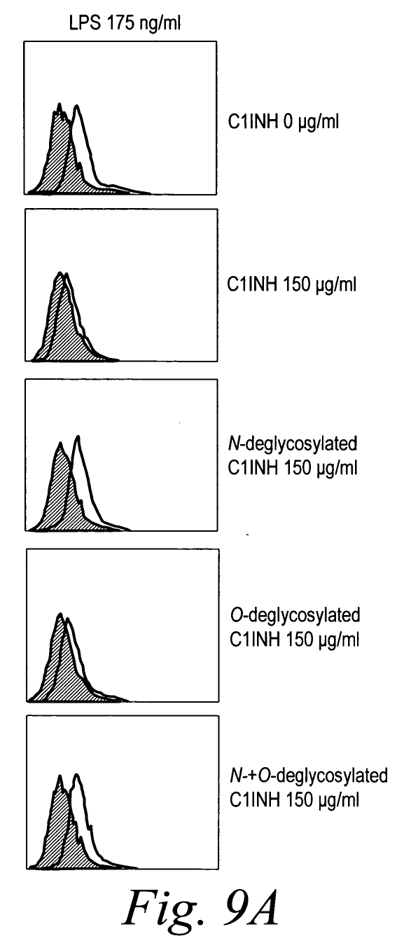

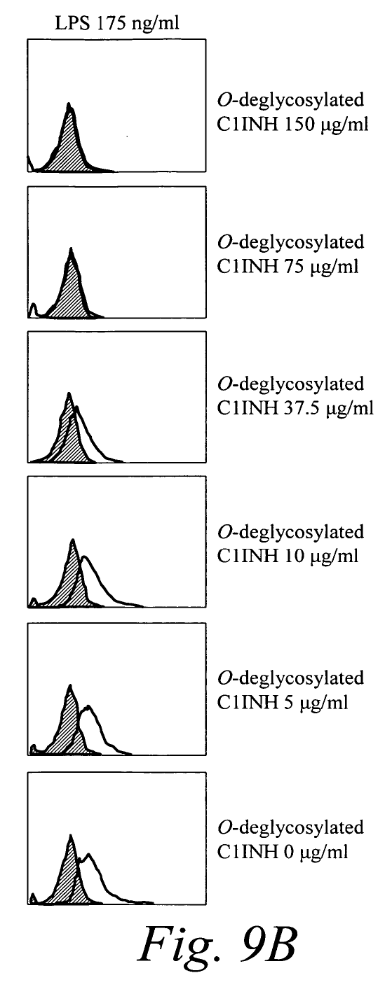

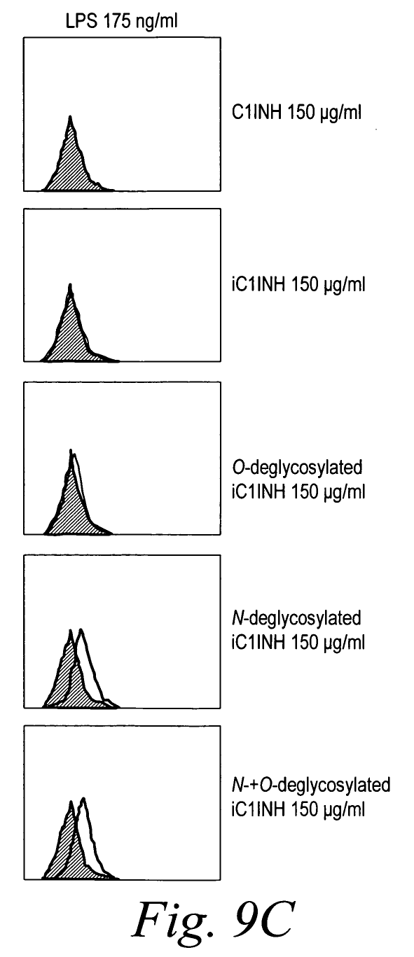

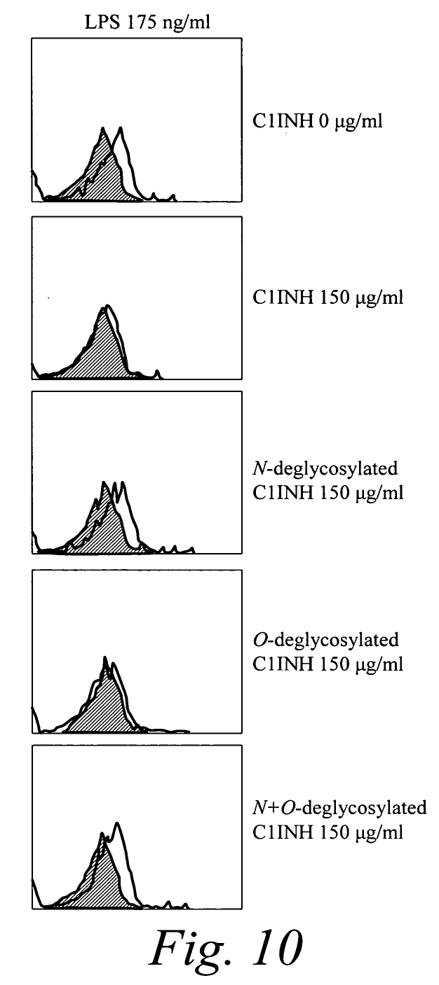

The finding that C1INH has the ability to block the binding of Salmonella typhimurium LPS to macrophages prompted further investigation of whether N— or O-linked glycosylation participates in this suppression of LPS binding. The ability of deglycosylated C1INH to inhibit LPS binding to RAW 264.7 macrophages in the presence of 10% FBS by FACS was tested. N-deglycosylated C1INH (150 μg/ml) lost the ability to suppress FITC-LPS binding to RAW 264.7 cells. However, removal of O-linked carbohydrate had no effect on the inhibition of LPS binding (FIGS. 9 A & B). C1INH in which the reactive center loop has been cleaved with trypsin (iC1INH) also inhibits the binding of LPS to RAW 264.7 cells. As with intact C1INH, removal of N-linked carbohydrate eliminates this activity while O-deglycosylation has no effect (FIG. 9C). In addition, whole human blood was incubated with LPS (175 ng/ml) in the presence of C1INH (150 μg/ml) and the three different forms of deglycosylated C1INH. N-deglycosylation eliminated the ability of C1INH to block the binding of LPS to human blood cells, while C1INH lacking O-linked glycosylation retained the ability to suppress LPS binding (FIG. 10).

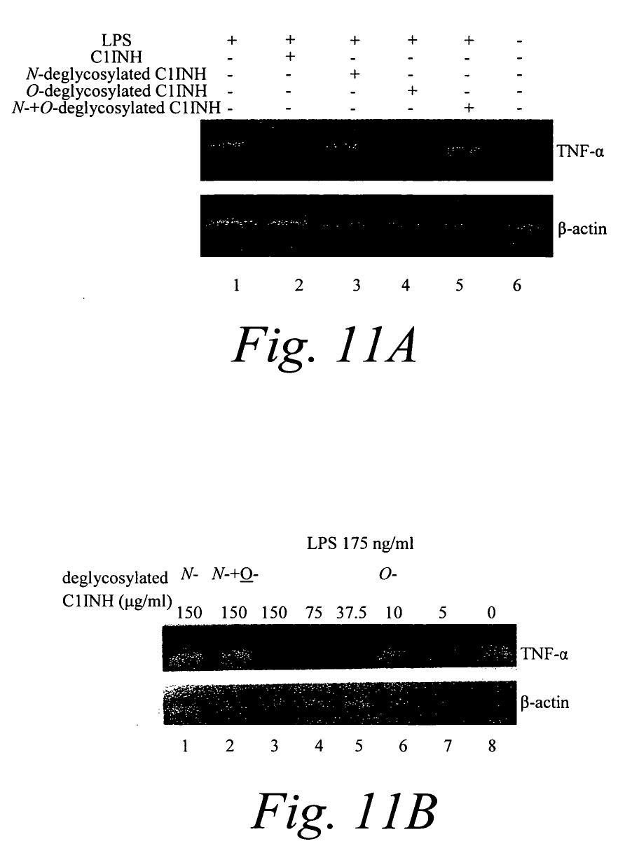

In addition, TNF-α mRNA expression induced by LPS was detected in RAW 264.7 cells using RT-PCR. LPS-mediated upregulation of TNF-α mRNA was completely suppressed following treatment with native plasma-derived C1INH (150 μg/ml) or with O-deglycosylated C1INH (150 μg/ml) (FIG. 11A). Similarly, C1INH with O-linked carbohydrate removed (150 μg/ml) also suppressed LPS-induced TNF-α mRNA expression by cells in whole human blood (FIG. 11B). However, removal of N-linked carbohydrate completely destroyed the ability of C1INH to inhibit LPS-mediated upregulation of TNF-α by both RAW 264.7 cells and by human peripheral blood cells (FIGS. 11A & 5B).

N-Linked Glycosylation of C1NH is Crucial for its Interaction with LPS.

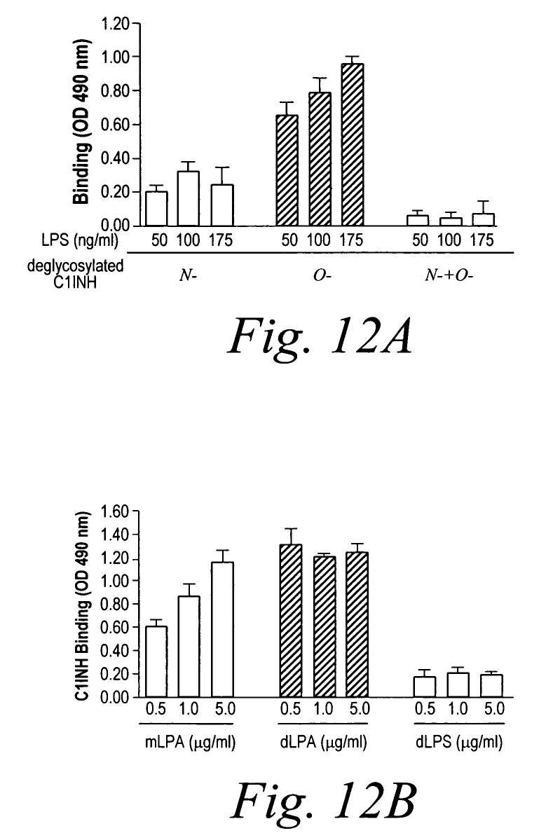

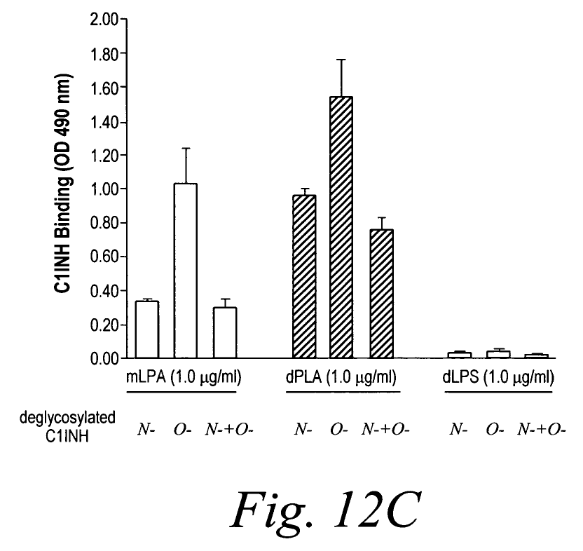

LPS is composed of two structural regions: the hydrophilic repeating polysaccharide of the core and O-antigen structures and the hydrophobic LPA domain (Ulevitch, R. J., and P. S. Tobias (1995) Annu Rev Immunol. 13:437-457). Virtually all the biological responses induced by LPS are dependent upon LPA (Grabarek, J., G. et al. (1990) J Biol Chem. 265:8117-8121; Rietschel, E. T., (1994) FASEB J. 8:217-225; Takada, H., and S. Kotani (1989) CRC Crit Rev Microbiol. 16:477-423). As expected from the experiments described above, deletion of N-linked or of N— and O-linked carbohydrate from C1INH completely eliminated its ability to bind to LPS, but the binding of O-deglycosylated C1INH (150 μg/ml) was unaltered in comparison with native C1INH (FIG. 12A). To further investigate the mechanism of interaction of C1INH with endotoxin, intact C1INH and the different forms of deglycosylated C1NH were analyzed for binding to immobilized mLPA, dLPA, or dLPS using an ELISA. Analysis of C1INH binding to mLPA (0.5, 1.0, and 5.0 ng/ml), dLPA (0.5, 1.0, and 5.0 ng/ml), and dLPS showed binding to both DLPA and mLPA, but very little binding to dLPS (FIG. 12B). Similarly, O-deglycosylated C1INH bound to both mLPA and dLPA (FIG. 12C). N-deglycosylated (and MO-deglycosylated) C1INH showed very little binding to mLPA. Both, however, did bind to dLPA, although not as well as did native or O-deglycosylated C1INH. These data indicate that N-linked glycosylation is essential to the interaction of C1INH with LPS. As with most other proteins that bind to LPS, LPA likely represents the primary structure in the LPS molecule that is recognized by C1INH.

Neither LPA nor dLPS Alter the Ability of C1INH to Complex with C1s.

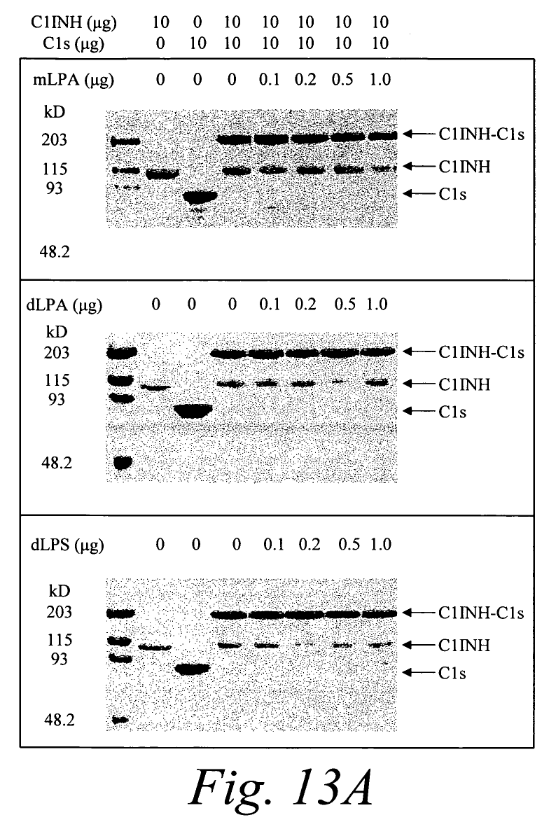

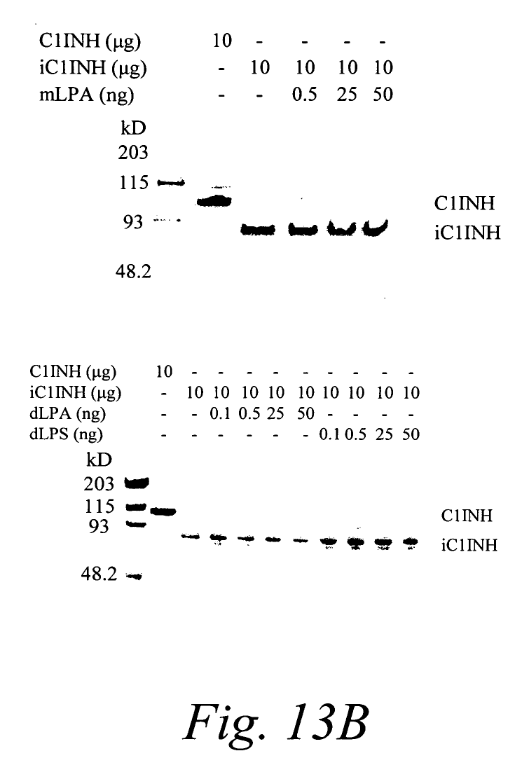

To examine whether mLPA, dLPA, and dLPS had an effect on the formation of C1INH-C1s complexes, native, active C1INH (10 μg) was incubated with mLPA, dLPA, and dLPS, respectively, followed by the addition of C1s (1 g). The C1INH treated with mLPA, dLPA, and dLPS retained the ability to form C1INH-C1s complexes (FIG. 13A). In addition, mLPA, dLPA, and dLPS did not interfere with the susceptibility of the reactive center loop to cleavage with trypsin (FIG. 13B).

Equivalents

Those skilled in the art will recognize, or be able to ascertain using no more than routine experimentation, many equivalents to the specific embodiments of the invention described herein. Such equivalents are intended to be encompassed by the following claims.

1. A method for treating spesis in a subject comprising administering to said subject an effective amount of a composition comprising a modified C1INH polypeptide, thereby treating sepsis in a subject.

2. A method for modulating the binding of LPS to a cell comprising contacting LPS with an agent which specifically binds LPS but does not inhibit activation of the complement system, thereby modulating the binding of LPS to a cell.

3. A composition comprising a modified C1INH polypeptide, wherein said modified C1INH polypeptide does not contain an intact serpin reactive loop.

4. The method of claim 1, wherein the composition reduces LPS-mediated inflammation in the subject.

5. The method of claim 1, wherein the composition inhibits LPS-induced TNF-α release in the subject.

6. The method of claim 1, wherein the composition inhibits binding of LPS to a cell in the subject.

7. The method of claim 1, wherein said modified C1INH polypeptide binds LPS.

8. The method of claim 1, wherein said modified C1INH polypeptide lacks an intact serpin reactive loop.

9. The method of claim 1, wherein said modified C1INH polypeptide comprises at least one mucin-like domain.

10. The method of claim 1, wherein said modified C1INH polypeptide comprises at least one tetrapeptide sequence comprising an amino acid sequence Glx-Pro-Thr-Thr.

11. The method of claim 1, wherein said modified C1INH polypeptide comprises amino acids 23-119 of C1INH, or an active fragment thereof.

12. The method of claim 1, wherein said modified C1INH polypeptide contains at least one intact N-linked carbohydrate.

13. The method of claim 12, wherein said intact N-linked carbohydrate is present at amino acid residues 25, 69 and/or 81 of SEQ ID NO:2.

14. The composition of claim 3, wherein said modified C1INH polypeptide binds LPS.

15. The composition of claim 3, wherein said modified C1INH polypeptide comprises at least one mucin-like domain.

16. The composition of claim 3, wherein said modified C1INH polypeptide comprises at least one tetrapeptide sequence comprising an amino acid sequence Glx-Pro-Thr-Thr.

17. The composition of claim 3, wherein said modified C1INH polypeptide comprises amino acids 23-119 of C1INH, or an active fragment thereof.

18. The composition of claim 3, wherein said modified C1INH polypeptide contains at least one intact N-linked carbohydrate.

19. The composition of claim 18, wherein said intact N-linked carbohydrate is present at amino acid residues 25, 69 and/or 81 of SEQ ID NO:2.


SEQUENCE LISTING (10 sequences)

SEQ ID NO: 1 - DNA, 1801 bp, Homo sapiens
ccagaagtttggagtccgctgacgtcgccgcccagatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctgggcagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagacctgcaggatcaggttagggcgagcgctacctctccagcctcagctctcagttgcagccctgctgctgcctgcctggacttgcccctgccacctcctgcctcaggtgtccgctatccaccaaaagggctcctgagggtctgggcaagggacctgcttctattagcccttctccatggccctgccatgctctccaaaccactttttgcagctttctctagttcaagttcaccagactctataaataaaacctgacagaccat

SEQ ID NO: 2 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 3 - DNA, 22 bp, Artificial sequence
  Primer
atgagcacagaaagcatgatcc

SEQ ID NO: 4 - DNA, 21 bp, Artificial sequence
  Primer
gaggccatttgggaacttctc

SEQ ID NO: 5 - DNA, 19 bp, Artificial sequence
  Primer
tggatgacgatatcgctgc

SEQ ID NO: 6 - DNA, 20 bp, Artificial sequence
  Primer
agggtcaggatacctctctt

SEQ ID NO: 7 - DNA, 30 bp, Artificial sequence
  Primer
atgagcactgaaagcatgatccgggacgtg

SEQ ID NO: 8 - DNA, 26 bp, Artificial sequence
  Primer
aggtccctggggaactcttccctctg

SEQ ID NO: 9 - DNA, 30 bp, Artificial sequence
  Primer
atggatgatgatatcgccgcgctcgtcgtc

SEQ ID NO: 10 - DNA, 26 bp, Artificial sequence
  Primer
agggtgaggatgcctctcttgctctg